(19) United States
(12) Patent Application Publication
(10) Pub. No.: US 2023/0386649 A1
(43) Pub. Date: Nov. 30, 2023

(54) SYSTEM AND METHOD FOR MULTIDIMENSIONAL COLLECTION AND ANALYSIS OF TRANSACTIONAL DATA

(71) Applicant: Memoro LLC, Tallahassee, FL (US)

(72) Inventors: Rand T. Lennox, Indianapolis, IN (US); Beecher C. Lewis, Tallahassee, FL (US)

(21) Appl. No.: 17/963,139

(22) Filed: Oct. 10, 2022

Related U.S. Application Data
(60) Provisional application No. 63/345,404, filed on May 24, 2022.

(51) Int. Cl. G16H 40/20; G06F 18/2413; G06F 3/04847; G06F 3/16; G06F 3/0482; G10L 17/22

(52) U.S. Cl. CPC G16H 40/20; G06F 18/2413; G06F 3/04847; G06F 3/167; G06F 3/0482; G10L 17/22

(57) ABSTRACT

The present disclosure provides an automated, integrated system and methods to enable process discovery, conformance, performance, and organization analyses in the provision of high-quality patient care management, patient education, patient engagement, and care coordination. The multimodal process mining system and methods allows capture and analyses of data relating to complex clinical workflows, process model extraction of patient care events, monitoring deviations by comparing model and data collection, social network or organizational mining, automated simulation of models, model extension, case prediction, and recommendations to improve conformance, performance, or process outcomes.

### CROSS-REFERENCE TO RELATED APPLICATIONS

This application claims priority benefit of U.S. Provisional Application Ser. No. 63/345,404, filed May 24, 2022, entitled “SYSTEM AND METHOD FOR MULTIDIMENSIONAL COLLECTION AND ANALYSIS OF TRANSACTIONAL DATA”; the entirety of which is hereby incorporated herein at least by virtue of this reference.

### FIELD

The present disclosure relates to the field of patient care management, patient education, patient engagement, and care coordination through automated process mining, model discovery, performance outcomes analyses, and presentation.

### BACKGROUND

Healthcare systems in different parts of the world face unprecedented challenges, such as constant and rapid changes in clinical processes in response to new scientific knowledge, and the provision of high-quality care with limited resources. However, healthcare can be more affordable, efficient, and effective with innovations. One method of innovation in clinical processes is the development and implementation of clinical pathways, also known as care pathways, critical pathways, or sometimes even as care programs or shared baselines. Clinical pathways are defined as complex interventions performed by a network of healthcare specialists for the mutual decision making and the organization of care for a specific patient group during a well-defined period sequenced on a timeline. In general, real-life clinical pathways are characterized by high flexibility, since all patients in need of the same treatment come with different comorbidities and complications, and involve complex decision-making due to their knowledge-intensive nature. Medical practitioners face uncertainty, unexpected outcomes, and complications during the treatment of patients with unique medical backgrounds and conditions. The inherent diversity of patients and hence care processes adds a further level of complexity which may be compounded by variability in the quality of the available data.

Many healthcare services have employed contemporary information systems to store a multitude of information on clinical pathway events in a structured manner. This information includes patient data in different forms such as numbers, text, images, audio, and logs. The data information would enable healthcare practitioners to make important medical decisions. An audit trail, also known as a trace or event sequence, for each specific pathway instance is precisely recorded in the event logs and represents the full diagnosis-treatment cycle of a specific patient. However, patient care processes are generally considered particularly challenging to describe and model in a realistic and comprehensive fashion. Methodologies for analyzing and describing processes have often been derived from manufacturing or service industries, where the analysis proceeds from routinely collected data; the procedure used is often referred to as “process mining.”

Process mining (PM) in a healthcare setting is gaining increasing focus due to the vast amounts of clinical data collected and stored in healthcare information system databases. PM analyses can be used to map and study clinical pathways. An automated discovery process enables a descriptive “process model” to be extracted (discovered) using an “event log” taken from a specific healthcare database. The complex nature of many healthcare processes means that the use of PM methods with healthcare datasets can be challenging. Equally, identifying the best PM methodologies for effectively extracting, “discovering” and visualizing the most relevant event data from such large and diverse healthcare datasets requires increasingly sophisticated algorithms and approaches. However, healthcare datasets can be complex to analyze, due to the variety of different medical codes used in claims databases (e.g., diagnoses, procedures, and drugs). Healthcare processes are complex, in part because they tend to exhibit significant variability, in terms of the vast diversity of activities that can typically be executed, subprocesses that can be executed simultaneously, and the influence of differences in the personal preferences/characteristics of patients, clinicians, workarounds (i.e., intentional deviations from prescribed practices) and other healthcare professionals. The combination of such factors tends to make almost all cases (e.g., a patient in a clinical process) different. In general, many healthcare processes are at least partially supported by Health Information Systems (HISs) such as Electronic Health Records (EHRs) systems that record data about the execution of processes or models in a healthcare organization. The pre-existing process model is often a protocol, guideline, or formally defined care pathway, and EHRs generally take the role of the event log.

HISs enable the study of clinical pathways using event logs composed of cases representing different process instances (e.g., the execution of a treatment process for a specific patient). Each case is composed of a sequence of events, where an event could refer to the completion of a particular activity in the treatment process. An event log typically records the following information for each event: (a) an identifier of each case, (b) the activities that each case included, and (c) a reference or timestamp to when each activity was performed. Besides this information, an event log can also contain information regarding the type of event (i.e., transaction type), the resource associated to an event, as well as other attributes regarding the activity or case. There are several challenges in processing EHR data including heterogeneous data, missing data, data quality, high-dimensionality, temporality which refers to the sequential nature of clinical events, sparsity in both medical code's representation and in timestamp representation, irregularly timed observations, biases such as systematic errors in data collection, and the distributed nature of healthcare providers. Moreover, it is recognized that some healthcare processes, specifically unplugged processes, are not directly supported by HISs. The presence of data quality problems can be attributed, at least partly, to the fact that event recording often still requires a manual action from clinicians or administrative staff. When an event log of a highly variable healthcare process is used to discover a control-flow model, control-flow discovery algorithms are likely to generate an unstructured model (i.e., spaghetti model). A challenge exists to capture high quality data describing patient pathways, medical events, provider-patient interactions, provider-provider interactions, performance quality, and outcomes in such HIS databases.

Data quality issues have a direct impact on the outcomes of healthcare process mining for healthcare. Healthcare processes captured through the patient's eyes can improve data quality. Capturing processes from the patient's perspective can help physicians to consider the full patient journey when making decisions and potentially uncover ways to enhance the patient's experience. For example, poor communication between patients and healthcare providers during transitions can lead to suboptimal outcomes (e.g., increased hospital re-admissions, being discharged to long-term care). Studies have shown that patients do not understand 50% of the information they receive while in the hospital, 80% forget what they were told, and 50% of the data “remembered” was recalled incorrectly. Inadequate patient and caregiver communication is a barrier to effective care transitions and hospital readmission reduction initiatives. Patients are sometimes overwhelmed with complex discharge instructions, leading to medication mismanagement or an unclear understanding of what signs and symptoms indicate that they should seek care from their healthcare provider. Reducing readmission rates through improved care transitions requires an evidence-based approach that incorporates adequate communication and optimized workflows. An essential factor in avoiding readmission is modification of a patient's behavior and providing information on preventative behavioral changes is an important part of the discharge process. An effective process structure is essential to systematically ensure appropriate levels of care for patients being discharged across shifts and care providers. These processes benefit both patient and care providers, and gaps in a process can have significant impact on a patient's health outcomes, and lead to future readmissions. Process mining allows practitioners and healthcare administrators to perform various analyses and aids them in understanding major deviations from clinical guidelines, clinical pathways, patient behavior, patient compliance, provider-patient interactions, risk management, and quality assurance, thus improving the quality and efficiency of patient care.

The need exists for an automated process mining system and methods to enable process discovery, conformance, performance, organization, and outcomes analyses in the provision of high-quality patient care management, patient education, patient engagement, and care coordination. The multimodal process mining system and methods allow analyses of complex clinical workflows, process model extraction of patient care events, monitoring deviations by comparing model and data collection, social network or organizational mining, automated simulation of models, model extension, case prediction, and recommendations to improve process outcomes.

### SUMMARY

The following presents a simplified summary of some embodiments of the invention to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

An aspect of the present disclosure is an automated and integrated system for process mining of healthcare data to enable process discovery, conformance, performance, organization, and outcomes analyses in the provision of high-quality patient care management, patient education, patient engagement, and care coordination. In various embodiments, the integrated system may comprise at least one process definition engine, process execution engine, ingestion engine, external connection engine, analysis engine, transaction data store, analysis data store, and a visualization engine. In various embodiments, the process definition engine provides a set of functions to define one or more processes existing within an organization. In a preferred embodiment, the process definition further comprises a content library, a taxonomy definition component and a process/workflow library. In various embodiments, the process execution engine may comprise at least one case management system for application configuration purposes. In various embodiments, the process execution engine may comprise one or more sub-components for the configuration of a process definition, external connection engine, case management system or combinations thereof and the like. In various embodiments, the ingestion engine may function to prepare one or more multimodal data sources, including but not limited to text, audio, and video for further processing, preferably by said analysis engine. In various embodiments, the analysis engine processes one or more data sources from a transaction data store. In various embodiments, the analysis data store may function to store one or more data from one or more data sources, preferably data produced by the analysis engine. In various embodiments, the visualization engine may function to combine the two or more outputs from the data analysis data store and/or the transaction data store to produce one or more analysis, report, or other visualizations from the output of the analysis engine. The multimodal process mining system enables the capture and analyses of multimodal data relating to complex clinical workflows, process model extraction of patient care events, monitoring deviations by comparing model and data collection, social network or organizational mining, automated simulation of models, model extension, case prediction, and recommendations to improve operational consistency, efficiency, precision, accuracy, analytics, costs, business, or process outcomes.

An aspect of the present disclosure is one or more automated methods for process mining of healthcare data to enable process discovery, conformance, performance, and organization analyses in the provision of high-quality patient care management, patient education, patient engagement, and care coordination. In various embodiments, one or more methods may comprise a process definition engine, process execution engine, ingestion engine, external connection engine, analysis engine, transaction data store, analysis data store and a visualization engine method. In various embodiments, the process definition engine method may comprise one or more steps to define one or more processes existing within an organization. In a preferred embodiment, the process definition engine method uses a content library, a taxonomy definition component, and a process/workflow library. In various embodiments, the process execution engine method may comprise at least one case management system steps for application configuration purposes. In various embodiments, the process execution engine method may incorporate the use of one or more sub-components for the configuration of a process definition method, external connection engine method, case management system method, or combinations thereof and the like. In various embodiments, the ingestion engine method may comprise steps to prepare one or more multimodal data sources, including but not limited to, text, audio, and video for further processing, preferably by said analysis engine. In various embodiments, the analysis engine method may comprise one or more steps for processing one or more data sources from a transaction data store. In a preferred embodiment, the one or more steps comprises one or more modified statistical, artificial, or machine learning methods, including but not limited to, clustering, near-neighbor, categorization, Apriori Item-Set, combinations thereof, or the like. In various embodiments, the analysis data store method may comprise one or more steps to store one or more data from one or more data sources, data preferably produced by the analysis engine. In various embodiments, the visualization engine may comprise one or more steps to combine the two or more outputs from the data analysis data store and/or the transaction data store to produce one or more analysis, report, or other visualizations from the output of the analysis engine. The multimodal process mining methods enable the automated capture and analyses of multimodal data relating to complex clinical workflows, process model extraction of patient care events, monitoring deviations by comparing model and data collection, social network or organizational mining, automated simulation of models, model extension, case prediction, and recommendations to improve operational consistency, efficiency, precision, accuracy, analytics, costs, business, or process outcomes.

An aspect of the present disclosure is a computer-implemented system configured for process mining of healthcare data to enable process discovery, conformance, performance, and organization analyses in the provision of high-quality patient care management, patient education, patient engagement, and care coordination. The computer system in accordance with the present disclosure may comprise systems and/or sub-systems, including at least one microprocessor, memory unit (e.g., ROM), removable storage device (e.g., RAM), fix/removable storage device(s), input-output (I/O) device, network interface, display, and keyboard. In various embodiments, the computer-implemented system may serve as a client enabling user access to the automated and integrated system and methods, locally or as a client, of a distributed computing platform or back-end server. In various embodiments, the general-purpose computing system may serve as a client enabling user access to the automated and integrated system and methods, locally or as a client, of a distributed computing platform or back-end server, via an administrative/navigator web interface. In various embodiments, the computer system in accordance with the present disclosure may comprise systems and/or sub-systems, including one or more desktop, a laptop, tablet, portable, or mobile phone computing device. In various embodiments, the computer-implemented system may comprise at least one process definition engine, process execution engine, ingestion engine, external connection engine, analysis engine, transaction data store, analysis data store, and a visualization engine. The multimodal process mining system enables the capture and analyses of multimodal data relating to complex clinical workflows, process model extraction of patient care events, monitoring deviations by comparing model and data collection, social network or organizational mining, automated simulation of models, model extension, case prediction, and recommendations to improve one or more operational consistency, efficiency, precision, accuracy, analytics, costs, business, or process outcomes.

An aspect of the present disclosure may comprise a mobile application (“app”) that enables a patient, care giver, or healthcare provider access to the automated or integrated system of said invention. A provider can use the app to select, configure, or use at least one function, including but not limited to, a diagnostic, intervention, prescription, education content, recommendation, scheduling, capture one or more multimodal data, forms, pre-surgical checklist, discharge instructions, rehabilitation, physical therapy instructions, relating high-quality patient care management, patient education, patient engagement, and care coordination. The app also allows a provider, doctor, nurse, healthcare manager, healthcare system management personnel, or patient to communicate, send, receive, or view the results of a process discovery, conformance, performance, organization analyses, improved operational consistency, efficiency, precision, accuracy, analytics, costs, business, or process outcomes.

Certain aspects of the present disclosure provide for a computer-implemented method comprising presenting, with a first processor communicably engaged with a display of a first client device, a first graphical user interface to a first end user, wherein the first graphical user interface comprises one or more interface elements configured to enable the first end user to configure at least one taxonomy comprising a plurality of data types for at least one user workflow; configuring, with the first processor, the at least one taxonomy in response to one or more user-generated inputs from the first end user at the first graphical user interface; presenting, with a second processor communicably engaged with a display of a second client device, a second graphical user interface to a second end user, wherein the second graphical user interface comprises one or more interface elements associated with the at least one user workflow; receiving, with the second processor via the second client device, a plurality of user-generated inputs from the second end user in response to the at least one user workflow, wherein the plurality of user-generated inputs comprises at least one input via the second client device and at least one voice input via a microphone of the second client device; processing, with one or both of the first processor and the second processor, the plurality of user-generated inputs according to at least one data processing framework to prepare a processed dataset comprising at least one audio file comprising the at least one voice input, wherein the at least one data processing framework comprises a speech-to-text engine configured to convert the at least one audio file to text data; analyzing, with one or both of the first processor and the second processor, the processed dataset according to at least one machine learning framework, wherein the at least one machine learning framework comprises a clustering algorithm configured to identify one or more attributes from the processed dataset and cluster two or more datapoints from the processed dataset according to the one or more attributes, wherein the at least one machine learning framework comprises a classification algorithm configured to analyze an output of the clustering algorithm to classify the one or more attributes according to a predictive strength for at least one quantitative outcome for the at least one user workflow, wherein the at least one machine learning framework comprises at least one Apriori algorithm configured to analyze an output of the classification algorithm to generate at least one quantitative outcome metric for the at least one user workflow; and presenting, with the first processor, the at least one quantitative outcome metric at the display of the first client device to the first end user.

In accordance with certain aspects of the present disclosure, the computer-implemented method may further comprise one or more steps or operations for generating, with the first processor, one or more recommendations for modifying or configuring one or more steps of the at least one user workflow according to the at least one quantitative outcome metric. The computer-implemented method may further comprise one or more steps or operations for algorithmically modifying or configuring, with the first processor, the one or more steps of the at least one user workflow according to the one or more recommendations. In certain embodiments, the classification algorithm comprises a naïve Bayesian algorithm. In certain embodiments, the clustering algorithm comprises a k-means++ clustering algorithm. The computer-implemented method may further comprise one or more steps or operations for analyzing, according to the at least one data processing framework, the at least one audio file to determine one or more speaker identity from the at least one voice input, wherein the at least one data processing framework comprises a speaker identification engine. The computer-implemented method may further comprise one or more steps or operations for analyzing, according to the at least one data processing framework, the at least one audio file to determine one or more degrees of sentiment for the one or more speaker identity. The computer-implemented method may further comprise one or more steps or operations for presenting, via the display of the first client device, the one or more recommendations for modifying or configuring the one or more steps of the at least one user workflow according to the at least one quantitative outcome metric. The computer-implemented method may further comprise one or more steps or operations for rendering, with the first processor via the display of the first client device, at least one graphical data visualization comprising one or more outputs of the at least one data processing framework and the at least one machine learning framework.

Further aspects of the present disclosure provide for a computer-implemented system comprising a client device comprising an input device, a microphone and a display; and a server communicably engaged with the client device, the server comprising a processor and a non-transitory computer-readable medium communicably engaged with the processor, wherein the non-transitory computer-readable medium comprises one or more processor-executable instructions stored thereon that, when executed, command the processor to perform one or more operations, the one or more operations comprising configuring at least one taxonomy comprising a plurality of data types for at least one user workflow; rendering an instance of a data capture application at the client device; presenting a graphical user interface of the data capture application to an end user at the display of the client device, wherein the graphical user interface comprises one or more interface elements associated with the at least one user workflow; receiving a plurality of user-generated inputs from the end user according to the at least one user workflow, wherein the plurality of user-generated inputs comprises at least one input via the input device and at least one voice input via the microphone; processing the plurality of user-generated inputs according to at least one data processing framework to prepare a processed dataset comprising at least one audio file comprising the at least one voice input, wherein the at least one data processing framework comprises a speech-to-text engine configured to convert the at least one audio file to text data; analyzing the processed dataset according to at least one machine learning framework, wherein the at least one machine learning framework comprises a clustering algorithm configured to identify one or more attributes from the processed dataset and cluster two or more datapoints from the processed dataset according to the one or more attributes, wherein the at least one machine learning framework comprises a classification algorithm configured to analyze an output of the clustering algorithm to classify the one or more attributes according to a predictive strength for at least one quantitative outcome for the at least one user workflow, wherein the at least one machine learning framework comprises at least one Apriori algorithm configured to analyze an output of the classification algorithm to generate at least one quantitative outcome metric for the at least one user workflow; and presenting the at least one quantitative outcome metric at the display of the client device to the end user.

Still further aspects of the present disclosure provide for a non-transitory computer-readable medium with one or more processor-executable instructions stored thereon that, when executed, command one or more processors to perform one or more operations, the one or more operations comprising configuring at least one taxonomy comprising a plurality of data types for at least one user workflow; rendering an instance of a data capture application at a client device; presenting a graphical user interface of the data capture application to an end user at a display of the client device, wherein the graphical user interface comprises one or more interface elements associated with the at least one user workflow; receiving a plurality of user-generated inputs from the end user according to the at least one user workflow, wherein the plurality of user-generated inputs comprises at least one input via an input device of the client device and at least one voice input via a microphone of the client device; processing the plurality of user-generated inputs according to at least one data processing framework to prepare a processed dataset comprising at least one audio file comprising the at least one voice input, wherein the at least one data processing framework comprises a speech-to-text engine configured to convert the at least one audio file to text data; analyzing the processed dataset according to at least one machine learning framework, wherein the at least one machine learning framework comprises a clustering algorithm configured to identify one or more attributes from the processed dataset and cluster two or more datapoints from the processed dataset according to the one or more attributes, wherein the at least one machine learning framework comprises a classification algorithm configured to analyze an output of the clustering algorithm to classify the one or more attributes according to a predictive strength for at least one quantitative outcome for the at least one user workflow, wherein the at least one machine learning framework comprises at least one Apriori algorithm configured to analyze an output of the classification algorithm to generate at least one quantitative outcome metric for the at least one user workflow; and presenting the at least one quantitative outcome metric at the display of the client device to the end user.

### BRIEF DESCRIPTION OF DRAWINGS

The above and other aspects, features and advantages of the present disclosure will be more apparent from the following detailed description taken in conjunction with the accompanying drawings, in which:

FIG. 1 is a block diagram of an automated and integrated system for process mining of healthcare data to enable process discovery, conformance, performance, and organization analyses in the provision of high-quality patient care management, patient education, patient engagement, and care coordination, in accordance with certain aspects of the present disclosure;

FIG. 2 is a block diagram of a process definition engine, in accordance with certain aspects of the present disclosure;

FIG. 3 is a block diagram of a process execution engine, in accordance with certain aspects of the present disclosure;

FIG. 3a is a screenshot of a possible case type list screen, in accordance with certain aspects of the present disclosure;

FIG. 3b is a screenshot of a possible case type actor role screen, in accordance with certain aspects of the present disclosure;

FIG. 3c is a screenshot of a possible case type topic and topic content screen, in accordance with certain aspects of the present disclosure;

FIG. 4 is a screenshot of a surgical discharge interaction type, in accordance with certain aspects of the present disclosure;

FIG. 5 is an implementation of a capture playback capability of a case management application, in accordance with certain aspects of the present disclosure;

FIG. 6 is a block diagram of an ingestion engine, in accordance with certain aspects of the present disclosure;

FIG. 7 is a block diagram of an analysis engine, in accordance with certain aspects of the present disclosure;

FIG. 8 is a pseudocode pattern for performing a k-means++ clustering, in accordance with certain aspects of the present disclosure;

FIG. 9 is a block diagram of the general steps required to perform a naïve Bayesian algorithm to determine a predictive strength, in accordance with certain aspects of the present disclosure;

FIG. 9a shows a pseudocode pattern for a naïve Bayesian algorithm, in accordance with certain aspects of the present disclosure;

FIG. 10 is a diagram for the example construction of an Item-sets of size k=4 using the Apriori Algorithm, in accordance with certain aspects of the present disclosure;

FIG. 11 is a block diagram of a general-purpose computer implemented system configured for process mining of healthcare data to enable process discovery, conformance, performance, and organization analyses in the provision of high-quality patient care management, patient education, patient engagement, and care coordination, in accordance with certain aspects of the present disclosure;

FIG. 12 is a block diagram of a mobile application in which one or more aspects of the present disclosure may be implemented;

FIG. 13 is a process-flow diagram of a computer-implement method, in accordance with certain aspects of the present disclosure; and

FIG. 14 is an illustrative embodiment of a computing device through which one or more aspects of the present disclosure may be implemented.

### DETAILED DESCRIPTION

It should be appreciated that all combinations of the concepts discussed in greater detail below (provided such concepts are not mutually inconsistent) are contemplated as being part of the inventive subject matter disclosed herein. It also should be appreciated that terminology explicitly employed herein that also may appear in any disclosure incorporated by reference should be accorded a meaning most consistent with the concepts disclosed herein.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. The present disclosure should in no way be limited to the exemplary implementation and techniques illustrated in the drawings and described below.

Where a range of values is provided, it is understood that each intervening value, to the tenth of the unit of the lower limit unless the context clearly dictates otherwise, between the upper and lower limit of that range and any other stated or intervening value in that stated range is encompassed by the invention. The upper and lower limits of these smaller ranges may independently be included in the smaller ranges, and are also encompassed by the invention, subject to any specifically excluded limit in a stated range. Where a stated range includes one or both endpoint limits, ranges excluding either or both of those included endpoints are also included in the scope of the invention.

As used herein, “exemplary” means serving as an example or illustration and does not necessarily denote ideal or best.

As used herein, the term “includes” means includes but is not limited to, the term “including” means including but not limited to. The term “based on” means based at least in part on.

As used herein, the term “process mining” is a set of tools to provide fact-based insights and to support process improvements built on process model-driven approaches and data mining of event data. The goal of process mining is to use event data to extract process-related information, e.g., to automatically discover a process model by observing events recorded by an information technology system.

As used herein, the term “discovery” is a method to obtain process models reflecting process behavior from, for example, an event or interaction log.

As used herein, the term “conformance” is the evaluation of a process model execution to detect deviations between the observed behavior in an event or interaction log and the process model.

As used herein, the term “enhancement” is a method to enrich and extend an existing process model using process data. One enhancement type is model repair, which allows the modification of a process model based on event or interaction logs. Another type is model extension, where information is added to enrich a process model with information such as time and roles.

Exemplary embodiments of the present disclosure provide an automated, integrated system and methods to enable process discovery, conformance, performance, and organization analyses in the provision of high-quality patient care management, patient education, patient engagement, and care coordination. The multimodal process mining system and methods allows capture and analyses of data relating to complex clinical workflows, process model extraction of patient care events, monitoring deviations by comparing model and data collection, social network or organizational mining, automated simulation of models, model extension, case prediction, and recommendations to improve process outcomes.

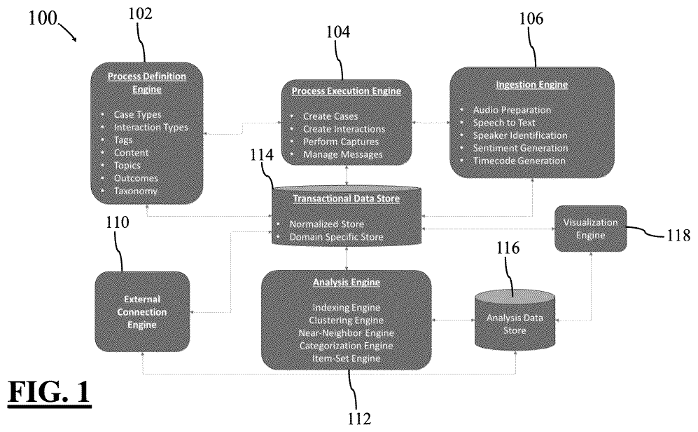

Turning now descriptively to the drawings, in which the same reference characters denote the same elements throughout the several views, FIG. 1 is a block diagram of an automated and integrated system 100 for process mining of healthcare data to enable process discovery, conformance, performance, and organization analyses in the provision of high-quality patient care management, patient education, patient engagement, and care coordination. In accordance with certain aspects of the present disclosure, the integrated system 100 may comprise at least one process definition engine 102, process execution engine 104, ingestion engine 108, external connection engine 110, analysis engine 112, transaction data store 114, analysis data store 116, and a visualization engine 118. In various embodiments, the process definition engine 102 provides a set of functions to define one or more processes existing within an organization. In a preferred embodiment, the process definition engine 102 further comprises a content library, a taxonomy definition component, and a process/workflow library. In various embodiments, the process definition engine 102 may comprise at least one case management system for application configuration purposes. In various embodiments, the process execution engine 104 may comprise one or more sub-components for the configuration of a process definition, external connection engine, case management system, or combinations thereof and the like. In various embodiments, the ingestion engine 106 may function to prepare one or more multimodal data sources, including but not limited to, text, audio, and video for further processing, preferably by said analysis engine 112. In various embodiments, the analysis engine 112 processes one or more data sources from a transaction data store 114. The transaction data store 114 may be a storage mechanism for all the data produced and/or consumed to execute the processes defined in the Process Execution Engine 104. This data store can take multiple forms (i.e., multimodal) depending on the needs of the item to be persisted. It can include database tables, a media management distributed file system, a “virtual store” that is accessed via real-time connection to an external system, etc. In an exemplary embodiment, the Transaction Data Store 114 comprises two main types of content, the Normalized Store, and the Domain Specific Store. The Normalized Store is the persistent storage for one or more items that are defined in a way that is usable independent of a business context or process. For example, case types are stored in the Normalized Store. It is understood that case types may be vastly different from one organization or industry to the next, but the concept of a case type and its attributes, ability to connect to taxonomies, ability to contain constituent interaction types, etc., may be defined in a way that is consistent across one or more industry and uses. Individual case types may vary, but the concept, purpose, and structure of a case type aspect will not. In various embodiments, all data in the Process Definition Engine 102 may be part of the Normalized Store and may be sourced outside of the store through the External Connection Engine 110. One non-limiting purpose of the External Connection Engine 110 may be to provide a data interchange between this system and any other external systems currently in use by the participants in an interaction or event. For example, the list of electronic medical records, patient data and registries, administrative data, claims data, health surveys, clinical data and the like are generally maintained in an electronic medical records (EMR) system (e.g., ORACLE CERNER, EPIC, etc.). The External Connection Engine 110 may provide a consistent means for performing one or more key services in accordance with certain aspects of the present disclosure, including: retrieving and updating information for the Domain Specific Store (e.g., patient lists, etc.); retrieving and updating any available information for the Normalized Store (e.g., an external system may provide functionality that helps define case types); and providing an automation point for the Process Execution Engine 104. The Process Execution Engine is the main subsystem that may be used by interaction participants, and the ability to reduce costs would be hampered if users were required to do “dual data entry”—that is, enter the same information into both the Process Execution Engine 104 and their existing business system. The External Connection Engine 110 provides a means for this data interchange to occur.

A key function of the External Connection Engine 110 may incorporate the use of one or more connectors. In various embodiments, a connector may be embodied as a computer-implemented method that permits the interchange of information between the system of the present disclosure and any external systems via one or more standardized interfaces. In various embodiments, one or more connectors can be defined in a manner that permits them to be multi-instance. For example, if a connector is developed to connect to a customer relationship management (CRM) system, then more than one instance of the connector can be executed for a large organization that may be using more than one instance of a CRM. In various embodiments, one or more standard interface may be defined using External Connection Engine 110 preferably through one or more customized software “widget” that can perform the interchange of data between Transactional Data Store 114 and any external system using technology that is appropriate to the external system. The second type of store is the Domain Specific Store. This is storage of data used during the process that is specific to the domain of the organization (e.g., healthcare system) conducting the business. For example, in a medical system, the Domain Specific Store may include information about patients, practitioners, medical procedures, etc. The Normalized Store provides an anchor point for gathering of information in a consistent manner to feed Analysis Engine 112. In various embodiments, the Domain Specific Store may provide a means of populating data in the Normalized Data Store. In a preferred embodiment, said Domain Specific Store provides most of the raw data needed to calculate one or more outcomes. This outcome data is a critical input to the Analysis Engine 112. In various embodiments, the Analysis Data Store 116 may function to store one or more data from one or more data sources, preferably produced by the Analysis Engine 112. In various embodiments, Analysis Engine 112 may comprise one or more artificial intelligence (AI), machine learning (ML) or data mining engine, including but not limited to, indexing, clustering, near-neighbor, categorization, or item-set engine. In various embodiments, the Visualization Engine 118 may function to combine the two or more outputs from the Data Analysis Store 116 and/or from the Transaction Data Store 114 to produce one or more analysis, report, or other visualizations from the output of the Analysis Engine 112. The multimodal process mining system enables the capture of the analyses of multimodal data relating to complex clinical workflows, process model extraction of patient care events, monitoring deviations by comparing model and data collection, social network or organizational mining, automated simulation of models, model extension, case prediction, and recommendations to improve operational consistency, efficiency, precision, accuracy, analytics, costs, business, or process outcomes.

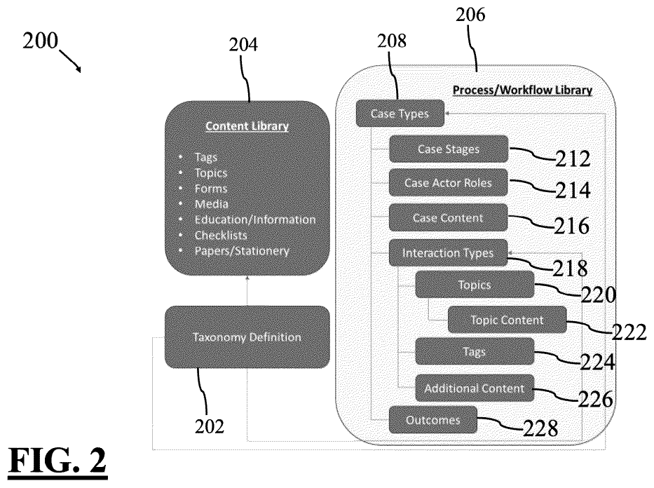

Referring now to FIG. 2 a block diagram of a process definition engine 200 is shown. In accordance with certain aspects of the present disclosure, process definition engine 200 may be embodied as Process Definition Engine 102 of FIG. 1. Process definition engine 200 may comprise one or more sub-system components that provide one or more set of functions to define the processes that currently exist in an organization. In a preferred embodiment, said processes are defined in a way that is flexible to model any service-oriented, clinical pathway, care pathway, or critical pathway workflow. In various embodiments, the said Process Definition Engine may further comprise a Taxonomy Definition 202 that enables an organization or user to define within the organization of one or more child components a Content Library 204, Process/Workflow Library 206, further comprising Case Types 208, and Interaction Types 218 according to a hierarchical structure for organization and classification purposes. In various embodiments, Taxonomy Definition 202 may function to structure one or more items, preferably referencing it in a manner that is understood by the business, organization, clinic, ambulatory surgical, emergency room, hospital ward, or healthcare system. In a preferred embodiment, one or more taxonomy may be defined in terms that reflect the business or the perspectives understood by that the organization. The ability to interpret results provided by the Analysis Engine 112 of FIG. 1 may be rooted in processes, terms, and structures familiar with the kind of business or functions of the organization. In various embodiments, Taxonomy Definition 202 may provide “roll-up” and/or “drill-down” capabilities for result analyses. In various embodiments, an organization may define more than one taxonomy tree for analytical purposes. In various embodiments, one or more trees may be hierarchical in nature, but more than one independent tree can be used to define independent taxonomies used in an organization. In various embodiments, each kind of item in Process Definition Engine 200 that is attached to the taxonomy may be (a) attached to more than one node in a single taxonomy tree; (b) attached to more than one taxonomy tree; and c) attached to leaf or parent nodes as needed. In various embodiments, each tree can be defined to any number of nodes or nesting depth of nodes. In an exemplary embodiment, one or more simple taxonomies may be defined as recorded in Table 1: Small Hospital Taxonomy.

TABLE 1
Small Hospital Taxonomy

| Specialty | Taxonomy |
|---|---|
| Orthopedic | Knee |
| | Hip |
| | Eye |
| | Spine |
| | Foot/Ankle |
| Cardiac | Heart |
| | Cardiovascular |
| Radiology | X-Ray |
| | CT |
| | MRI |
| Urgent Care | Emergency |
| | Walk-In Clinic |
| Family Practice | Preventative Care |
| | Chronic Management |

With respect to sub-system Content Library 204, the content library may store one or more multimodal data sources or information that may be used in the execution of a business, organization, clinical pathway, or critical pathway workflow or process. In various embodiments, the one or more multimodal data may include the following.
1) Media content comprising audio, video, images, or animations that are used during the execution of the process. In various embodiments, the media content may be training videos, animated images, photos, audiobooks, combinations thereof, or the like.
2) Educational/Information content comprising “read only” material that is used in the execution of the process. In various embodiments, said content may include, but is not limited to, handouts, links to web sites, brochures, information sheets, textbooks, illustrations, e-books, combinations thereof, or the like.
3) Checklists comprising one or more lists of items that are completed as part of executing a workflow or process to transition from one or more stages or events.
4) Forms comprising a generic term for the data collection instrument that is performed as part of the process. In various embodiments, said forms may include, but are not limited to, data entry completed on a computer system, PDF forms, spreadsheets, documents, block diagrams, combinations thereof, or the like.
5) Papers and Stationery comprising items used in the process for capturing handwritten information. In various embodiments, said papers and stationery may be “blank pages” (e.g., Yellow Lined Legal Paper) or block diagrams to be annotated during a conversation (e.g., a doctor may want to write on a block diagram of a knee to describe a surgical procedure to a patient.).
6) Topics comprising one or more agenda items that are discussed or covered during human-to-human interactions (e.g., patient-practitioner discussion, patient-nurse discharge instructions, etc.). When arranged together in a specific order, they describe the planned flow of an interaction. In various embodiments, the different business or workflow contexts, example topics may include “General Overview,” “Wrap up and Next Steps,” “Medications,” “Homework,” etc. It is understood that topics may represent the “proactive” part of an interaction, items that are planned to be covered during the interaction or event.
7) Tags comprising items that occur during an interaction or that will likely occur, but if or when they will occur is unknown. In various business or workflow contexts, tag examples may include “Call Doctor” (e.g., a patient should call the doctor if certain conditions are observed), “Activity Limitation” (e.g., a nurse just mentioned that the information currently being discussed represents an activity that the patient should curtail, avoid or cease), “Confidential” (e.g., a person mentioned that the information currently being discussed should be kept private). In various embodiments, said tags may represent a “reactive” part of an interaction, which may or may not happen, can happen multiple times in the conversation, or when they will happen cannot be predicted ahead of time.

With respect to sub-system Process/Workflow 206, the process and workflow library may contain information about the processes and workflows that results in one or more human-to-human, patient-doctor, patient-nurse, doctor-nurse, doctor-doctor, or administrator-workforce interactions. In various embodiments, one or more items in the process and workflow library are linked to the taxonomy either through connection to the content library or explicit connection in the process definition. In a preferred embodiment, the Process and Workflow Library 206 is configurable or customizable thus enabling a business or organization to define said items in any way relevant to their process needs.

An aspect of the present disclosure is a Process and Workflow Library 206 that may comprise one or more of elements 208-228, described in more detail below.

Case Types 208 are the root of the Process Library 206. Each Case Type 208 defines one or more kinds of processes that are performed by the business or organization. The term “Case” herein is defined as a generic term for one or more discrete business processes that have a defined start and a defined end as well as a workflow for task completion. Some illustrative examples include in a medical system, a case type may be a non-limiting surgical procedure, chronic condition, or in family practice the case could be a patient. Further illustrative examples include in a healthcare system, a case type may be a non-limiting chronic condition (e.g., a Diabetes Case, Heart Failure, etc.). This case would be a long-lasting case with an end point defined by discharge from care (e.g., the patient changed doctors) or the patient's mortality. In various embodiments, case types are linked to the one or more taxonomies and taxonomy nodes to facilitate analysis in the Analysis Engine 112 of FIG. 1.

Case Stages 212 represent the natural flow of a case from beginning to end. In a surgical procedure, for example, case stages may include Screening, Pre-Op, Surgery, Post-Op, Discharge and Follow Up procedures, combinations thereof, or the like.

Case Actor Roles 214 define the roles that are performed by human participants in the case. In a medical surgical case, for example, roles may include but are not limited to a Patient, Patient Home Care Giver, Surgeon, Anesthesiologist, Surgical Nurse, Discharge Nurse, Medical Technician, Physician's Assistant, or Business Office Representative.

Case Type Content 216 is the list of items from the content library that may be used on a given case type. In various embodiments, said content may be needed to advance a case from beginning to end.

Interaction Types 218 comprises one or more human-to-human interactions that occur during, for example, a medical encounter, a clinical workflow, a clinical pathway, or a critical pathway. In a surgical procedure case, for example, Interaction Types 218 may include, but are not limited to, Diagnosis Appointment, Screening Appointment, Pre-Op Appointment, Post-Op Appointment, Discharge Meeting, Follow Up Appointment, combinations thereof and the like.

Interaction Type Topics 220 are the ordered list of Topics drawn from the Content Library 204 that indicate which topics will be covered in what order for a given interaction type. For example, a surgical discharge meeting may include the following topics: What to Expect After Surgery, Care Instructions, Medication, Precautions, Physical Therapy, Next Appointment, combinations thereof and the like.

Interaction Type Topic Content 222 is the list of Content drawn from the Content Library 204 that is used when covering a specific topic 220 on a specific interaction type 218. For example, when discussing Medications during a surgical discharge appointment, an information sheet may be used to describe one or more medication, their purpose, their dosing requirements, an image of each medication, combinations thereof, or the like.

Interaction Type Tags 224 are Tags drawn from the Content Library 204 that are expected to be used for a given interaction type 218. For example, for a medical procedure, tags may include, but are not limited to, Call Doctor, Activity Limitation, Discharge or Transition of Care Instructions, Medication Instructions, Warning Signs and Symptoms, Emergency Response, combinations thereof and the like.

Interaction Type Additional Content 226 is content 216 that is not used during an interaction 218 but may be used by participants in preparation for the interaction or used after the conclusion of the interaction. For example, for a surgical interaction this may include forms to fill out prior to arriving for surgery and discharge instructions to have on hand after the surgery.

Outcomes 228 are the possible results of a case type 208, case stage 212, or interaction 218. Outcomes 228 may include, but are not limited to, a definition of a) a means to measure them through a formula or procedure using information in the transactional data store 114 of FIG. 1 and b) whether a business goal, clinical, surgical, diagnostic, or therapeutic aspect is to minimize or maximize the outcome. In various embodiments, one or more outcomes 228 may define how success or failure is measured for a case, case stage, or interaction, and every case, case stage, or interaction may have multiple measurable outcomes (e.g., Patient Satisfaction, Cost of Acquisition, Mean Time to Closure, Profit Margin, Readmission Rate, etc.)

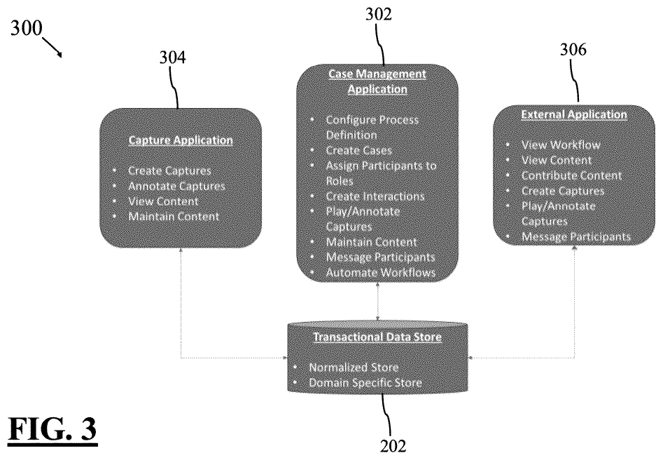

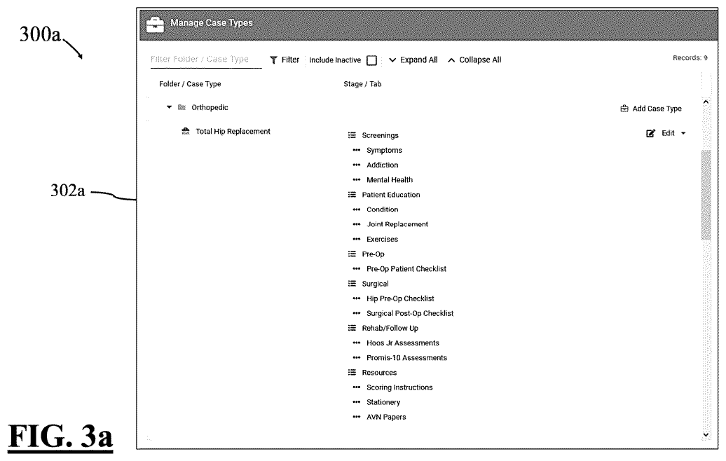

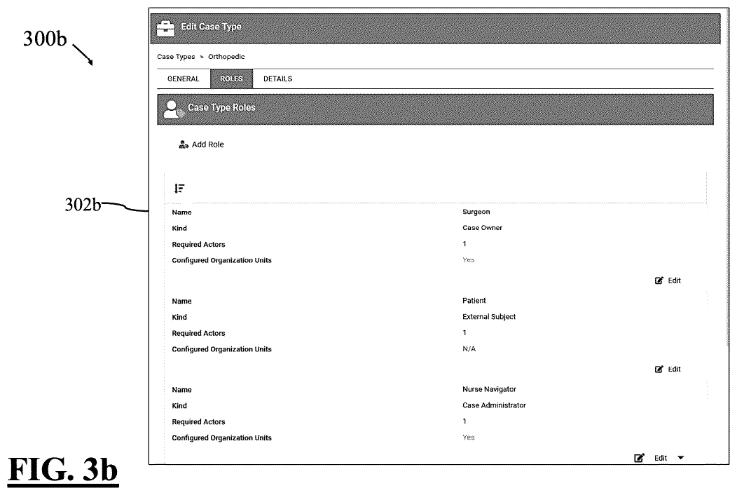

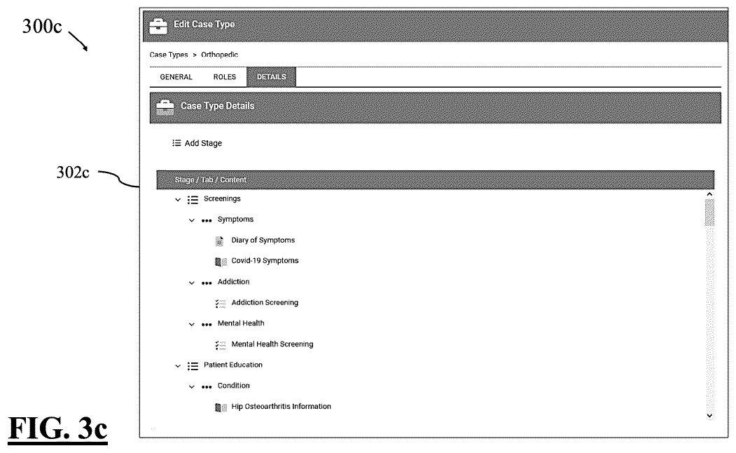

Referring now to FIG. 3, a block diagram of a process execution engine 300 is shown. According to various embodiments, process execution engine 300 is equivalent to Process Execution Engine 104 of FIG. 1. Process execution engine 300 may provide a primary set of software tools that enable capturing of human-to-human interactions while limiting the impact of this capture on participants in the interaction. In various embodiments, said process execution engine may process one or more case which are one or more instances of a case type (e.g., Case Type 208 of FIG. 2) containing assigned actors (e.g., Case Actor Role 214 of FIG. 2), case content (e.g., Case Content 216 of FIG. 2), interactions (e.g., Interaction Type 218 of FIG. 2), a private message thread, and lifecycle management. In various embodiments, said case actor may be a person assigned to a case performing a role defined in said process definition engine. An important aspect for each case actor role assignment is that each may be designated as an “internal” actor or an “external” actor. An internal actor is a person employed (e.g., nurse, doctor, surgeon, etc.) or otherwise engaged to deliver services on behalf of the organization or healthcare system. An external actor is a participant (e.g., patient) in the case who is receiving the services of the organization. This classification is used by said analysis engine when determining behaviors or process steps that influence outcomes. In various embodiments, said process execution engine may process one or more interaction, human-to-human meeting, or human-to-human encounter that occurs during a case. In various embodiments, one or more people may participate in an interaction, and they may or may not be physically present (for example, some via telephonically). Depending on the business or organization, an interaction may be referred to as a “Meeting,” “Appointment,” “Session,” etc. Each interaction is assigned to one or more interaction types as defined in the said process definition engine, with the type(s) reflecting the purpose of the interaction. For example, an interaction type of “Surgical Discharge” can be used for a patient discharge at 3:00 pm on Nov. 22, 2022. The interaction type defines the agenda, tags, topics, and content used by all appropriate case actors on the case in conducting the surgical discharge. In various embodiments, said process execution engine may capture one or more time-series recording comprising one or more multimodal information interchanged during at least one interaction. During a capture, each time a piece of content is used by the participant, the use is time stamped or event logged. Additionally, if the capture is being audio recorded, the timestamp to synchronize with the audio is also recorded. In various embodiments, one or more single pen stroke, selection of an agenda topic, tag tapped, checkbox checked, form field filled in, annotation made, etc. is timecoded when performing the capture. A single interaction may have one or more captures. In a surgical discharge example, both the patient and the discharge nurse may be performing a capture of the interaction. Furthermore, in that same interaction, there may be several medical professionals who visit the patient at various times during the discharge (e.g., the surgeon, physical therapist, discharge nurse, etc.) Each of these participants may have an independent capture for their portion of the interaction. Each capture may have its own independent interaction type, since the material covered by each “visit” could be different or heterogeneous depending on the participant (e.g., the topics and materials used by the physical therapist could be different than those of the surgeon). In a preferred embodiment, one or more content used in the interaction should be the same content that the user would otherwise use in performing their job. The forms preferably should be non-limiting forms to be fill out, non-limiting required checklists, non-limiting educational materials or presentations, and a non-limiting agenda (ordered topics) with topics needed to cover during an interaction. In this manner, work that is completed during the interaction is “actual work,” and no additional work is introduced; however, by implementing aspects of the present disclosure to perform this work the timecoding of the work is performed at a granular level—with every significant touch on a screen, stroke on the keyboard, or word spoken during the audio recording (if one is performed) of a computing device or platform. In accordance with certain aspects of the present disclosure, process execution engine 300 comprises a case management application component 302, a capture application component 304, and an external application component 306. In various embodiments, case management application component 302 may comprise a system that enables a properly authorized user to configure the items in the Process Definition Engine 102 of FIG. 1, configure connectors for the External connection Engine 110 of FIG. 1, create or maintain cases, and view, annotate and share captures.

Still referring to FIG. 3, case management application 302 may comprise one or more sub-component used to open and close cases, work with content on a case, communicate with case participants via the private message thread, create interactions for the case, and view, play, share, and annotate captures that occur during an interaction on the case. In various embodiments, a user may or may not use a case management sub-component system. In various embodiments, with sufficient integration through the External Connection Engine 110 of FIG. 1, all the functions of the case management subsystem may be performed by an existing user system. In a preferred embodiment, the information managed by the case management system may be accessible to the Analysis Engine 112 through the Transactional Data Store 114 of FIG. 1.

Referring now to FIG. 3a, a screenshot 300a of a possible case type list screen is shown according to various embodiments. In various embodiments, a process definition subcomponent enables an organization to define and configure all the items needed by the Process Definition Engine 102 of FIG. 1. In an exemplary embodiment, screen 302a shows a case type configuration for an orthopedic total hip procedure.

Referring now to FIG. 3b, a screenshot 300b of a possible case type actor role screen is shown according to various embodiments. In various embodiments, a process definition subcomponent enables an organization to define and configure all the items needed by the Process Definition Engine 102 of FIG. 1. In an exemplary embodiment, screen 302b shows a case type configuration for an orthopedic procedure whereby case type actors include a surgeon, a patient, and a nurse navigator.

Referring now to FIG. 3c, a screenshot 300c of a possible case type topic and topic content screen is shown according to various embodiments. In various embodiments, a process definition subcomponent enables an organization to define and configure all the items needed by the Process Definition Engine 102 of FIG. 1. In an exemplary embodiment, screen 302c shows a case type topic and content configuration for an orthopedic procedure whereby case type topics and contents include, but are not limited to, screening, symptoms, diary of symptoms, addiction, addiction screening, mental health, mental health screening, patient education, condition, and information on hip osteoarthritis. In various embodiments, one or more similar screens may be developed to configure all other aspects of the defined for the Process Definition Engine 102 of FIG. 1. In a similar manner, one or more External Connection Engine Configuration component is used to install, configure, and activate/deactivate connectors with external systems.

Another aspect of the present disclosure is a Case Management Application 302 of FIG. 3 providing messaging capabilities. Messaging is a capability that enables case participants to communicate messages regarding a case. These messages can be text as well as binary documents and media. In various embodiments, a graphical user interface may be provided by the system or by an external messaging application that is integrated through the External Connection Engine 110 of FIG. 1. In addition, multiple sources of messaging information may be incorporated through the said external connection engine. For example, an organization may integrate feeds from third-party applications, such as ORACLE CERNER, EPIC, customer service IVR recordings, email content, and the like. Messaging provides the ability to incorporate as much information regarding the communications among case participants as is possible into the analysis.

Another aspect of the present disclosure is that the benefits to the business for use of the system and methods described herein should be shared by both the organization and its users. In a preferred embodiment, users of the system may see no perceived productivity penalties for using the system but productivity gains through workflow automation. In various embodiment, the following automation points are important to the Analysis Engine 112 of FIG. 1.

Reminders: Since the system includes forms, checklists, and educational media, each of these items may result in a task assignment that can be tracked, (e.g., checklist items need to be completed, educational items need to be reviewed, and forms need to be filled out). The system provides a system for sending reminders to case participants that their tasks are due. The timing of the viewing of the reminder, the amount of time before the item referenced is completed, and the number of times a reminder is needed for an item until it is completed are used as inputs to the analysis engine.

Workflow Triggers: The external integration engine may implement workflow triggers, as appropriate. Workflow triggers are functions that either start a workflow stage, mark the completion of a workflow stage, or record the outcome of a workflow stage. Automating triggers for workflow stages is preferable to manually updating for two reasons: 1) It reduces the data entry effort for participants, and 2) it provides more accurate data to the analysis engine for actual workflow timings since there will not be lags between the time a stage is started or completed and the time these events are recorded in the system.

Automated Annotations: As stated previously, additional weight is placed on annotations during analysis because they represent matters that case participants regard as significant during the execution of the process. While the capability must be provided to allow participants to manually annotate captures, it is desirable to also have the system find and annotate items in the capture to which they would otherwise manually assign significance themselves (i.e., process discovery).

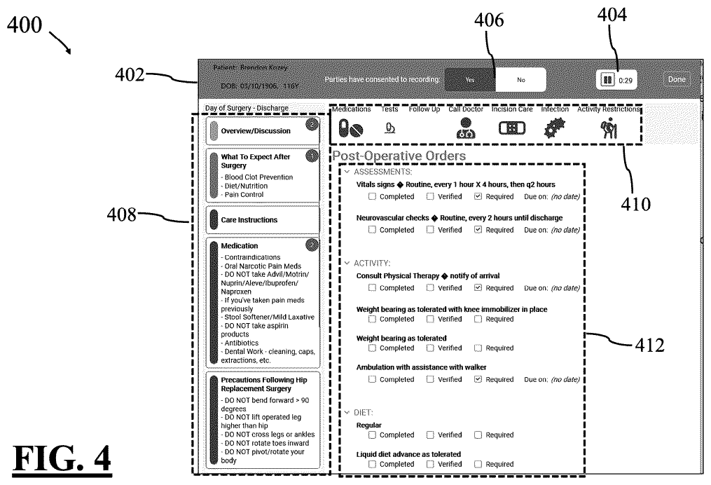

Yet another aspect of the present disclosure is a capture application that enables a case actor (e.g., 214 of FIG. 2) to capture fine-grained process execution data while performing their usual work. In a preferred embodiment, said capture application is the primary mechanism by which said data is obtained for further analysis. Referring now to FIG. 4, a screen shot 400 of a surgical discharge interaction type is shown, according to various embodiments. In accordance with certain aspects of the present disclosure, the capture application may be implemented on a mobile tablet or a mobile computing platform and presented to a user using a graphical user interface (GUI). In a preferred embodiment the capture application screen may be divided into four major sections. In one section, at least one control section 402 enables the management of an audio recording 404 and a recording consent 406 of the participant. In a second section, an agenda section 408 on the left shows the topics to be covered and in what order during the interaction. In a third section, a tag section 410 below the control section 402 enables a user to tap or click on one more tagged item as they occur. In a fourth section, a content area 412 displaces one or more content used by the participants during the interaction. In preferred embodiments, one or more tags and topics shown are based on the configuration defined in the Process Execution Engine 104 of FIG. 1 for the given interaction type. The content shown is linked to the selected topic and the user is presented with the content they need for each agenda item. Any work performed on this screen (e.g., filling out a sample checklist) is both timecoded and completed as part of the capture. The checklist options are persisted, and the user does not need to key them in again in another system. If audio is being recorded, each action performed is indexed to both the real-world time and the audio time clock. For example, tapping on a topic is timestamped to the audio, checking a check box is timestamped to the audio, etc.

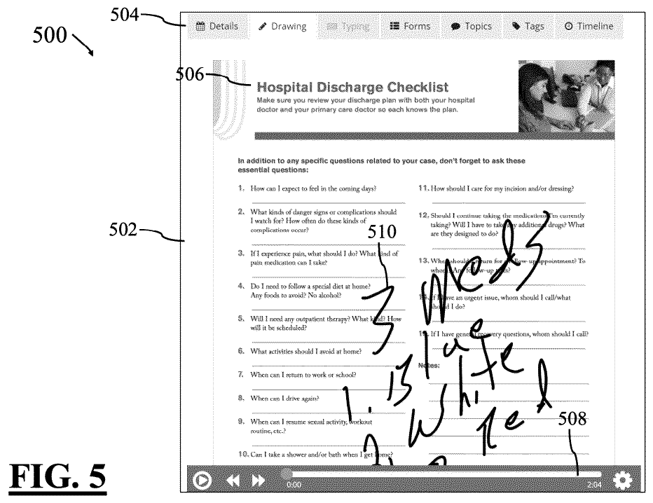

Referring now to FIG. 5, an implementation of a graphical user interface 500 of a capture playback capability of a case management application is shown. According to certain aspects of the present disclosure, the case management application may be embodied as Case Management Application 302 of FIG. 3 and may comprise one or more View, Annotate, and Share Captures component that provides the ability to “play back” captures or data recording of an interaction or an event that are performed using a capture application. In various embodiments, the said components may be implemented on a mobile tablet or a mobile computing platform and presented to a user using, for example, a graphical user interface 502. In various embodiments, the application provides a menu bar 504 enabling a user to choose from a list of non-limiting functions; for example, Details, Drawing, Typing, Forms, Topics, Tags, or Timeline. In an exemplary embodiment, a hospital discharge checklist 506 is presented to a user, which may include a nurse, a doctor, or a patient. In various embodiments, one or more action, interaction, or event between a patient, nurse, and doctor is timecoded as part of the capture, and a user may tap on one or more timecoded aspect in the capture to immediately move to portion 508 (e.g., 2:00 minute recording mark) of a conversation, action, interaction, or event. In various embodiments, portion 508 may comprise one or more action or event, including but not limited to, pen strokes 510, entry of form fields, tags tapped, agenda topics started, combinations thereof, or the like. An aspect of the present disclosure is to provide a playback capability that enables productivity gains to internal case participants. Multiple studies have shown the inability for participants in conversations or interactions to retain this information for any extended period. For example, numerous healthcare studies between healthcare professionals and patients reveal that 40-80% of information shared during their interactions is forgotten. In the case of critical patient healthcare information being shared, not only is more than half of the shared information forgotten, but half of what is remembered is remembered inaccurately or incorrectly. Additionally, discharge instructions provided to patients in multiple healthcare settings are critical to patients understanding and performing patient-specific care instructions that are mandatory to obtain a desired healthcare recovery or outcome. In an exemplary embodiment, providing patients and their caregivers with a reliable playback method to repeatedly review and recall all the various detailed care instructions represents one benefit of the capture-playback capability of the present disclosure. In addition to the ability to play back, captures can also be “annotated.” In various embodiments, one or more annotations may comprise simple “bookmarks” in the audio recording of capture that can be added with additional text or media files attached. These can be used to provide additional contextual information for other viewers of the capture. Additionally, these annotations, like all other data, are included in the feed to the Analysis Engine 112 of FIG. 1. Annotations are afforded a high level of significance in Analysis Engine 112 of FIG. 1 since they represent places in the audio where case participants found special meaning.

Another aspect of the present disclosure is an external application by which external case actors can participate in one or more cases from one or more different organizations. In certain embodiments, the external application may comprise or be embodied as External Application 306 of FIG. 3. In accordance with certain aspects of the present disclosure, External Application 306 of FIG. 3 comprises one or more following characteristics.

View Cases: If a participant is an actor on more than one case, the participant can view and access each case in which they play a role. The system assures that there is a single consolidated view for cases from different organizations if more than one organization is using the system. View Workflow: The external application allows a participant to see all the steps in the case workflow, the progress of each step, and any actions they are required to take, if any, as an actor in the case to move the case forward towards completion.

Work with Content: All case content that is externally accessible may be viewed or completed and submitted to the organization. For example, the external actor may view training materials, complete, and submit forms, mark off items on checklists, review informational videos, etc. In a preferred embodiment, all actions by one or more user, interaction, or event are timestamped for later reporting and analysis in the Analysis Engine 112 of FIG. 1. This tracking is very fine grained. For example, when the user views an informational video, the system will track each time they start and stop the playback, each time they jump forward or rewind, where exactly in the video each of these actions occurred, what portions of the video were viewed or not viewed, and in what order they were viewed.

Messaging/Contribute Content: External actors may message other case participants and contribute additional content to the case through message attachments. The content of these messages is used by the said analysis engine in the same manner as messages from internal participants.

View/Annotate Captures: The external actor can view captures for any interaction for which they were a participant. Like the capabilities afforded internal users, external users can use the timecoding in the capture to immediately jump to portions of the capture that are of immediate interest to them. As users are viewing captures, each “touch” is timestamped and recorded for later analysis. Like internal participants, external participants may also annotate captures with information that is significant to them.

Create Captures: The external actor can create their own captures of an interaction on a case, similar to the capture application used by internal case actors. As with external actors, every touch and keystroke are timestamped and synchronized with the audio recording (if any) to facilitate recall and to provide fine-grained process execution data to the analysis engine.

Share Captures: The external actor may share captures that they have created or can view with any other actor on the case. Subject to permissions from the organization, they may also electronically share these captures with other people who are not actors participating in the case.

The playback and sharing capabilities are one example of how certain aspects of the present disclosure provide productivity gains to external process participants. For example, in healthcare, patient interactions with clinical staff occur in multiple settings, and are critical to patient understanding, education, compliance and outcomes. Clinical staff are acutely aware that patients will forget most of the information shared with them or their caregiver networks, leading to actual patient complications, wasted clinical staff time, added costs per patient episode, decreased patient satisfaction, unnecessary consequences such as Emergency Room visits, missed appointments and avoidable hospital readmission, and ultimately a reduction in patient quality of care and outcomes. Studies show deficiencies in patient comprehension, recall and retention during these interactions. Having the ability to capture entire interactions between clinical staff and patients, and then selectively share these interactions with the patient, caregivers, even with other healthcare professionals within a given patient's continuum of care such as specialists, primary care doctors, or nursing home staff would be invaluable to the ultimate outcomes observed by said patients.

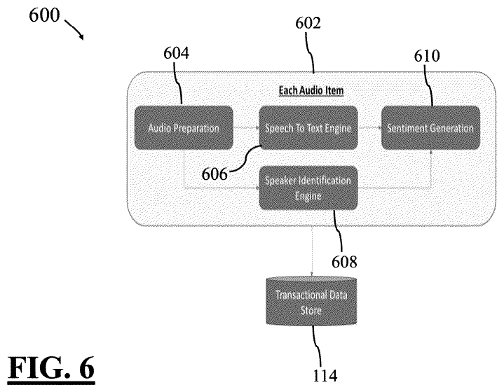

Yet another aspect of the present disclosure is an ingestion engine 602 for the preparation at least one multimodal information data or media for further processing the analysis engine 112 of FIG. 1. Referring now to FIG. 6, a block diagram 600 of an ingestion engine 602 is shown. In accordance with certain aspects of the present disclosure, engine ingestion 602 comprises an Audio Preparation stage 604, a Speech-to-Text Generation stage 606, a Speaker Identification stage 608, and a Sentiment Analysis stage 610. In a preferred embodiment, one or more outputs from each stage are stored in the Transactional Data Store 114 of FIG. 1. In various embodiments, the audio preparation stage 604 serves to prepare one or more incoming audio data in a manner that preferably will produce more accurate results in future stages with one or more of the following functions.

Audio Decompression: Converts all incoming audio, regardless of format (MP3, AAC, etc.), to Raw PCM audio.

Audio Joining: When a participant starts and stops recording during a capture, multiple audio files are created. Audio joining creates a single audio for the capture from these multiple audio files.

Noise Reduction: Reduces background noise (such as wind noise) in the audio.

Frequency Filtering: Applies low and high pass filters to reduce audio frequencies outside of the human “spoken word” range of 150 Hz-8000 Hz.

Dynamic Range Normalization: Adjust dynamic range so that all audio portions are constrained to a range of 0.85-0.98 of maximum gain. Gain is applied or reduced as needed in various parts of the audio to maintain a consistent dynamic range throughout the audio.

Recompression: Recompresses all processed audio into a standard format and media container.

In accordance with certain aspects of the present disclosure, Speech-to-Text Generation stage 606 converts the audio conversation into text using at least one of the following functions.

Language Detection: Samples the audio to determine language or languages used during the conversation to create a natural language map for the audio.

Language Model Detection: Samples the audio for the need to include special language models (e.g., Medical Terminology, Legal Terminology, etc.) in various points in the audio. Updates the language map and map weightings with this information.

Text Transcription Generation: Uses the prepared language model map to perform speech-to-text translation.

Transcription Alignment: Aligns the generated speech to text transcription to the audio timeline.

In accordance with certain aspects of the present disclosure, the Speaker Identification stage 608 creates a map of “who is speaking when” in the conversation with at least one of the following functions.

Speaker Separation: Generates a timed map of speaker changes in the conversation as well as areas where multiple speakers are talking at the same time.

Speaker Voice Print Mapping: Uses the speaker changes and isolates audio according to the map to attempt to match the speaker with a known speaker voice print. When a match is found, it assigns a known speaker to the section of audio. When a match is not found, it appends the segment to a matching voice print candidate for later positive identification as voice prints are updated.

Speaker Id Alignment: Aligns the generated speaker map to the audio timeline.

In accordance with certain aspects of the present disclosure, Sentiment Analysis stage 610 utilizes the output from one or more prior said stages to determine the sentiment and sentiment changes of the conversation for each speaker during the conversation. It also determines sentiment and sentiment changes for the whole conversation. “Sentiment” may be defined as a participant's emotional response to the conversation. Sentiment is measured as a categorization of emotion as well as an emotional intensity. Multiple sentiments may be generated for the same portion of audio for the same speaker (e.g., fear and anger may be present at the same time, which is quite different than fear and hope being present at the same time). Note that sentiment analysis does not attempt to categorize the emotional content as “positive” or “negative”—it merely determines the sentiment being presented and the relative intensity of that sentiment at a given point in time. This is since in some cases, what may be normally perceived as a “negative” sentiment may in some circumstances be a desired sentiment.

An aspect of the present disclosure is an analysis engine that functions to receive and process data from the Transaction Data Store 114 of FIG. 1 and uses a staged set of processes to produce data sets that indicate what changes can be made to the process or to participant behaviors to improve the frequency of desired outcomes across one or more user workflows. It should be noted that the analysis engine does not suggest how these changes should be implemented. It only discovers that there is a given likelihood that a certain set of changes, when implemented, will increase the frequency of desired outcomes. For example, one change could be that a key indicator of success is that external participants (customers, patients, clients, etc., depending on the service business) exhibit a high degree of empathy at a certain stage in the process. This means that the analysis engine has determined that this condition must exist to “move the needle” towards better outcomes. An organization's response to this insight may include: 1) train staff in better ways to elicit an empathetic response from patients; 2) change the desired patient profile to “weed out” patients early in the process that do not present this response—thereby saving time by disengaging with “bad patients” early and thus saving costs; 3) in situations where it is not possible to “weed out” “bad patients”—e.g., a medical emergency room—put extra protections in place since the likelihood of an undesirable outcome is known to be increased; 4) change KPIs, metrics, and projections to accommodate the better understanding of process “realities”—in other words, avoid holding staff accountable for things beyond their control; and 5) Any other reasonable adjustment to policies or procedures derived from this knowledge.

It should also be noted that there is also value in changes that are not discovered by the analysis engine. By the nature of algorithms used, the analysis engine creates item-sets that are known to lead to increased desirable outcomes. These algorithms also have the effect of showing that any suggestion not in the final item-set will likely not have an impact on the frequency of desired outcomes, or at least will not have as great of an impact on outcomes as the items in the list. The ability to avoid wasted efforts on improvements that will have little to no impact also saves time and money for a business.

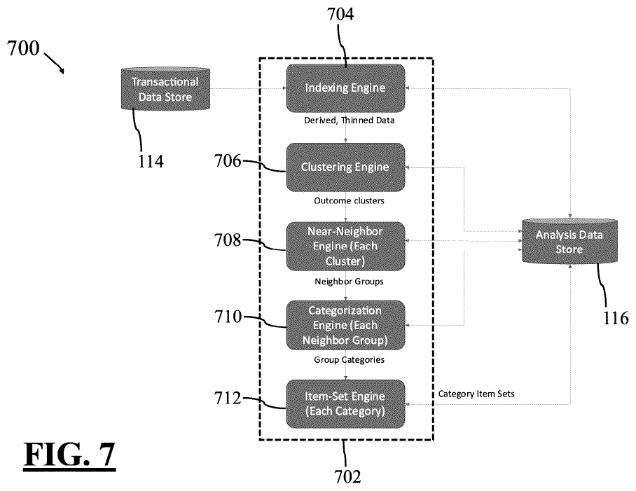

Referring now to FIG. 7, a block diagram 700 of an analysis engine 702 is shown. In accordance with certain aspects of the present disclosure, an analysis engine 702 comprises an Index Engine 704, a Cluster Engine 706, a Near-Neighbor Engine 708, a Categorization Engine 710, and an Item-Set Engine 712. Analysis engine 702 may be configured to process data from the Transaction Data Store 114 of FIG. 1 starting with Index Engine 704. In various embodiments, Index Engine 704 executes at least one of the following functions: 1) group the data into clustering groups based on time series, case type and interaction type; 2) winnow data into semantically useful data; 3) create derived data from the raw data; and 4) apply semantic weighting. Each of the above functions is described in more detail, as follows.

### Create Clustering Groups

Creating clustering groups is a process of grouping the input data by case type and by interaction type, then taking the result and only selecting a data subset that represents a reasonable period for analysis. The reasonable period will vary by case type and is set during configuration; however, it should generally be set to the period matching 25% of the annual lifecycle of the case. For example, if 10,000 cases of that type are opened and closed in a year, then the subset factor would be 2,500. If 10,000 cases of that type would be opened and closed in 5 years, then the subset factor would be 500. This will ensure that a reasonable sample size of interactions and captures will be gathered for subsequent stages.

Winnow Data into Semantically Useful Data

Data winnowing is applied to text transcriptions produced by the speech-to-text portion of the ingestion engine and any other conversational text in the case (e.g., messages, emails, etc.) Winnowing of conversational text takes two forms: Sematic Reduction and Word Stemming. Semantic reduction is the process of removing “stop words” from the conversational text data. Stop words are words that are used in conversation or writing that are linguistically necessary but do not contribute significantly to sematic meaning. For example, in the phrase “The young child ran down the street to see the dog” could be reduced to “Young child ran down street see dog”—a reduction from 11 words to 7, or a 36% reduction. When this technique is applied to all the conversational text, the processing time for subsequent stages is dramatically reduced. It should be noted that stop word reduction is not quite as simple as removing words that match a list. For example, the word “and” may have significance or not depending on the context. Therefore, the sematic reduction process removes stop words based on the semantic context in which the word is used in the language model by weighting the significance of the words in context and removing those that fall beneath a given threshold.

Word Stemming is the process of normalizing word “forms” so that words with different forms are translated to a common form. For example, the words “party,” “partying,” “partied,” and “parties” have the single word stem of “parti-”; the words “run,” “running,” and “ran” have a single word stem of “run-”. By reducing words to word stems, processing effort is reduced in further stages and different words with similar meanings are treated as a single semantic concept.

Create Derived Data from the Raw Data

For much of the time-coded data, the useful information for analysis is not in the instance of the data, but rather the timing, number, and order of transitions in the data. For this information, the indexing engine creates derived data. For example, assume a meeting agenda in a capture has 6 topics. Further assume that each topic was covered, and 2 of the topics were revisited. The item of interest for analysis is not when each topic was touched, but rather in what order they were visited, how many times they were visited, and most importantly how much time was spent on each topic (calculated from the time a topic was touched until the time another topic was touched). Additionally, meeting pauses are recorded by the capture application (for example, a lunch break was taken during a meeting). These meeting pauses must be removed from the timing calculations for the meeting. The Indexing Engine 704 uses the raw information to create a variety of derived data (e.g., how much time was spent on a topic) and adjusts for meeting pauses. It also time-aligns multiple captures for the same interaction (two or more individuals recorded the same meeting on their own device) to consolidate or aggregate overlapping data points where they exist.

### Apply Semantic Weighting

In various embodiments Indexing Engine 704 may function to apply sematic weighting to the data for the Cluster Engine 706. The primary targets of semantic weighting are annotations and emotionally intensive transcription areas. These items and transcription areas are tagged as more significant before the data is passed to the said clustering engine and will therefore be assigned a higher weight when clusters are generated. In various embodiments, Cluster Engine 706 processes one or more time-series case-interaction groups and creates clusters that represent grouping of related factors—in other words—to create clusters where items in the cluster show a statistically significant correlation. Some of these clusters will contain data points that represent outcomes, and some will not. The clusters that contain at least one data point representing an outcome are the clusters of interest that will be passed to the next stage. Clusters without an outcome data point are excluded from the next stage since they represent interaction activity that is highly correlated, but not highly correlated with one or more outcomes. Clustering is performed on each case-interaction group from the indexing engine. “Cluster-able” attributes are the attributes of data from each case in the group as described elsewhere in this submission. In one embodiment, clustering is performed using the k-means++ clustering algorithm.

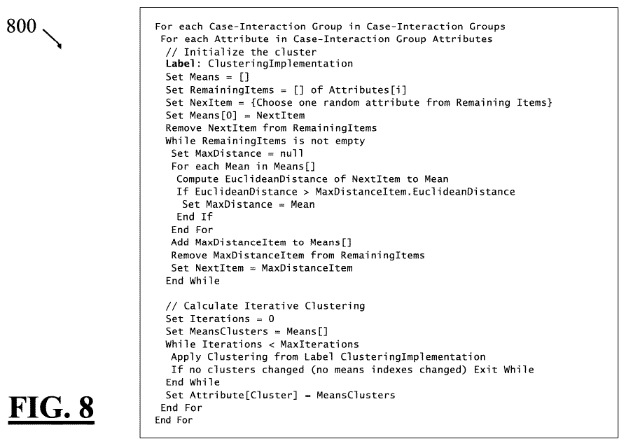

Referring now to FIG. 8, a pseudocode pattern 800 for performing a k-means++ clustering is shown. In accordance with certain aspects of the present disclosure, a k-means++ clustering method comprises one or more steps executed in one or more iterations or computing loops. In an exemplary embodiment, for each Case-Interaction Group and for each Attribute a cluster is initialized and labeled as Clustering Implementation. In a first step, the means of a cluster and the remaining items are set within one or more array with the remaining items set as an array of attributes. In various embodiments, one or more random attributes from Remaining Items(RI) may be set equal to a NextItem(NI). In a second step, the Means of an array is set equal to a said NextItem(NI). In various alternative steps, one or more NextItem(NI) may be removed from the RemainingItems(RI). In a third step, while RemainingItems(RI) are not empty, a MaxDistance(MD) is set equal to null. In a fourth step, for each mean in the Mean array, a EuclideanDistance(ED) of one or more NextItem(NI) is calculated to a Mean. In various embodiments, if ED is greater than MaxDistanceltem.Euclideandistance then set MD=Mean. In various embodiments, said steps are executed while RemainingItems is not empty using one or more computing language executable by a computing device or platform. In various embodiments, additional steps are performed by the Clustering Engine 706 of FIG. 7. In various embodiments, in a fifth step, MD is added to the array of Means. In a sixth step, MD may be removed from RI. In a seventh step, MD is set equal to NI. In various embodiments, one or more subsequent steps are performed, preferably to calculate one or more Iterative Clustering steps. In step eight, one or more iterations are set to zero. In step nine, one or more MeansClusters(MC) is set equal to one or more Means array. In various embodiments, one or more said steps stemming from step eight or nine is executed sequential or in parallel to apply clustering from one or more label clustering implementation while one or more iterations are less than a maximum number of iterations. In various embodiments, if no clusters are changed or no means indices are changed, the said steps are exited or terminated. In a subsequent step ten, one or more cluster attributes are set equal to the means of a clusters. Once the clusters are generated, clusters which contain one or more outcome attributes are passed to the Near-Neighbor Engine 708 of FIG. 7.

In accordance with certain aspects of the present disclosure, the purpose of Near-Neighbor Engine 708 of FIG. 7 is to examine all the attributes in a cluster and to determine the predictive strength for an outcome of each attribute in each cluster from stage 2 on a scale of −1.0 (no predictive strength) to +1.0 (extremely high predictive strength). Attributes from each stage with a predictive strength of less than 0.4 are discarded from the analysis in said Categorization Engine stage 710 of FIG. 7. Since only clusters with an outcome from the Cluster Engine stage 706 of FIG. 7 are included, outcome attributes are not included in this analysis (each outcome would have a predictive strength of 1.0 since they are selected-in exclusively). The overall goal is to winnow the number of attributes that are passed to the Categorization Engine stage 710 of FIG. 7 to those factors that are predictive (strength 0.4 or greater) of the outcome or outcomes in each cluster. In various embodiments, the Near-Neighbor Engine 708 of FIG. 7 may comprise one or more AI, ML, or statistical algorithm to determine one or more predictive strengths. In an exemplary embodiment, the Near-Neighbor Engine 708 of FIG. 7 uses a naïve Bayesian algorithm to determine predictive strength.

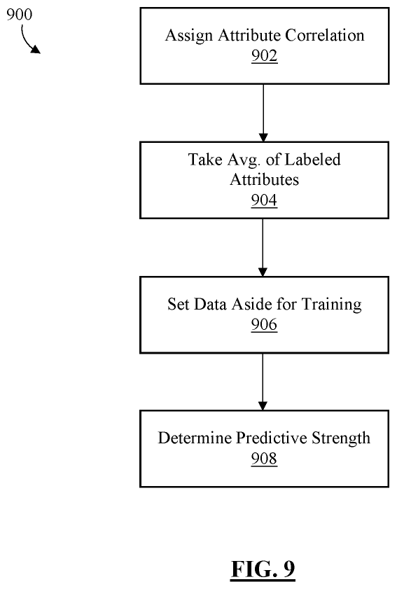

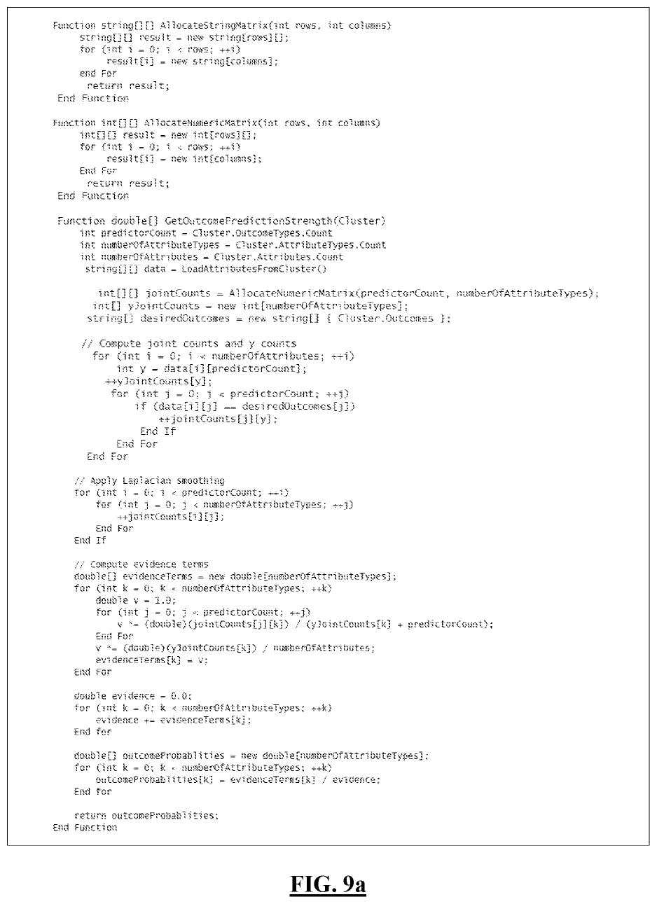

Referring now to FIG. 9, a block diagram 900 of the general steps required to perform a naïve Bayesian algorithm to determine predictive strength, in accordance with various aspects of the present disclosure, is shown. In a first step 902, one or more conformance or performance outcome data received by External Connection Engine 110 of FIG. 1 is provided from the Transaction Data Store 114 of FIG. 1. A correlation of +1 is assigned to each attribute present in the source with a positive outcome and a correlation of −1 to each attribute from said source with a negative outcome. In a second step 904, one or more labeled attributes are then averaged to produce an overall score, per attribute, on the range of −1.0 to +1.0. In a third step 906, said data is set aside for training or process mining modeling purposes since it represents actual conformance or performance outcomes derived from the selected attributes. In a fourth step 908, the Near-Neighbor Engine 708 of FIG. 7 examines one or more attributes of at least one cluster to determine the predictive strength using a naïve Bayesian algorithm. In accordance with certain aspect of the present disclosure, a pseudocode pattern for the naïve Bayesian algorithm may be as shown in FIG. 9a.

An aspect of the present disclosure is Categorization Engine 710 of FIG. 7 that processes one or more per-cluster outcome predictive strengths calculated by Near-Neighbor Engine 708 of FIG. 7 to determine the predictive strength of outcomes across all clusters containing the desired outcome. In various embodiments, said Near-Neighbor Engine determines which attributes strongly contribute to the desired conformance or performance outcomes contained in an individual cluster, whereas Categorization Engine 710 of FIG. 7 determines which attributes strongly contribute to desired outcomes across multiple clusters when a given outcome is contained in multiple clusters. In various embodiments, Categorization Engine 710 of FIG. 7 may use Naïve Bayesian categorization to achieve this purpose, but the input and training set for the stage are different. In a similar manner to said Near-Neighbor Engine 708 of FIG. 7, actual outcomes from the Transactional Data Store 114 of FIG. 1 are used, but the predictive values assigned are not −1.0 to +1.0. Instead, the predictive values for seeding the training set are the predictive values calculated by the Near-Neighbor Engine 708 of FIG. 7. Also, since only values with a predictive strength of 0.4 or higher are returned from the Near-Neighbor Engine 708 of FIG. 7, any attribute that does not meet that threshold is eliminated from both the data input and the training data for the Categorization Engine 710 of FIG. 7. While prior stages work with smaller and smaller sets of data, in Categorization Engine 710 of FIG. 7 stage, the data set is expanded back out to all clusters which may result in long and intensive computing time and power. In a preferred embodiment, Categorization Engine 710 of FIG. 7 uses the following strategy to overcome the computational challenges afforded by the present disclosure. The algorithm for this stage is identical to the said naïve Bayesian algorithm with the following changes; predictorCount is set to the number of outcomes across clusters; numberOfAttributeTypes is set to the count of AttributesTypes with a predictive strength of 0.4 or higher; numberOfAttributes is set to the count of Attributes with an AttributeType that has a predictive strength of 0.4 or higher; data is set to an array that is the union of data from multiple clusters from the Near-Neighbor Engine 708 of FIG. 7 stage that only contains attributes that have a 0.4 or higher predictive strength.

The output of this classification stage is the predictive strength of attributes when considered across multiple clusters. This output is fed as the input for the Item-Set Engine stage 712 of FIG. 7.

An aspect of the present disclosure is an Item-Set Engine 712 of FIG. 7 capable of finding one or more discovering the combination of attributes that when taken together as a set will provide the greatest impact on improving desired conformance or performance outcomes. It has been discovered that conventional approaches can be computation resource intensive. Each item in the item set raises the compute requirements by a power of two. For example, finding the set of the 2 most impactful changes is a compute time multiplier of 2\hat{ }2=4; finding the set of the 8 most impactful changes is a compute time multiplier of 2\hat{ }8=256; finding the set of the 20 most impactful changes is a compute time multiplier of 2\hat{ }20=1,048,576, etc. Therefore, the present disclosure proposes the following solution to manage the computational challenges of this stage. The output from the Categorization Engine 710 stage of FIG. 7 is winnowed to only include AttributeTypes with a predictive strength of 0.7 or higher. Calculations on the Categorization Engine 710 stage of FIG. 7 are done on a per outcome, per Case Type and per Interaction Type basis. Computationally, this approach allows for parallel processing of each type to reduce overall processing time. The Item-set limit should be limited to the number of changes a business or organization can reasonably implement in a 90-day window, typically 2-8 items. This has the added advantage of allowing new item sets to be calculated based on changes to the model as these changes are implemented—in other words, implementing the proposed changes will affect outcomes, which would then affect the model for calculating additional changes in the next 90-day business cycle.

An aspect of the present disclosure are various methods for conformance or performance outcomes discovery. In various embodiments, Item-Set Engine 712 of FIG. 7 may employ one or more algorithms. In a preferred embodiment, Item-Set Engine 712 comprises an Apriori Item Set algorithm. In various embodiments, said modified Apriori Item Set algorithm initiates all frequent item-sets of size k=1 of individual items that meet a predetermined threshold (e.g., those with a predictive value of 0.7 or higher) in a list of transactions or interactions. In various embodiments, said transactions may include non-limiting ingested interaction captures (e.g., with topics, tags, speech-to-text transcriptions, speaker id, events, sentiment analysis results, etc.) or other ingested information (e.g., gathered from the External Connection Engine 110 of FIG. 1). Then, the construction process iteratively adds frequent item-sets of size k=2, 3, 4, etc. until no new frequent item sets are found.

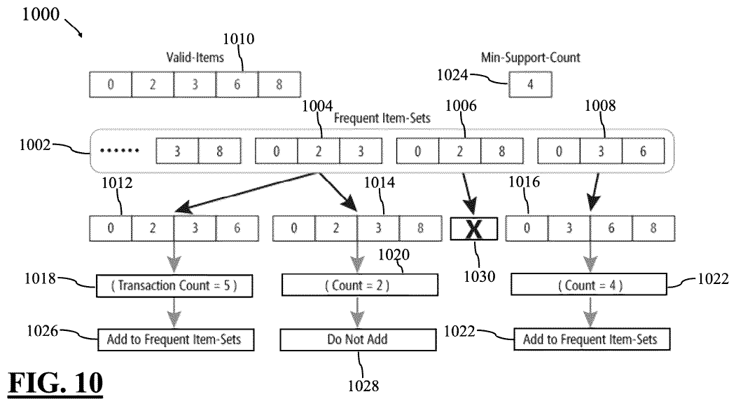

Referring now to FIG. 10, a diagram 1000 for the example construction of an Item-sets of size k=4 is shown. The construction requires the maintenance of a list 1002 of frequent item-sets of all sizes. In this example, there are shown three frequent item-sets, 1004 (0,2,3), 1006 (0,2,8), and 1008 (0,3,6). The method also maintains a list 1010 of items that are valid at a given point in time. In this example, there are shown five valid items: [0, 2, 3, 6, 8]. Valid items processed by Item-Set Engine 712 of FIG. 7 in this example may comprise one or more output AttributeTypes from the prior stage (e.g., those with a predictive value of 0.7 or higher). The valid items to construct frequent item-sets of size k=4 are the distinctness in all frequent item-sets of size k−1=3. In various embodiments, the method scans each frequent item-sets 1004, 1006, and 1008 of size k=3. For each said item-sets, a new candidate 1012, 1014, and 1016 of size k=4 is generated. For example, the fourth item of candidate 1012 can be filled in with a valid item. The method assumes the items within an item-sets are always stored in order so that the possible fourth item can be either a 6 or 8 in this case. In various embodiments, each new candidate 1012, 1014, or 1016 is examined at a transaction count 1018, 1020, or 1022 step to count how many times it occurs in the transactions list. If the transaction count meets a Minimum-Support-Count 1024 then the candidate is added (step 1026) to the list of frequent item-sets. The Minimum-Support-Count 1024 processed by Item-Set Engine 712 of FIG. 7 in this example may comprise the number of factors to consider for predetermined number-of-day business cycle, preferably but not limited to a 90-day business cycle. If the transaction count is below a Minimum-Support-Count 1024 then the candidate is not added (step 1028) to the list of frequent item-sets. This method greatly reduces computing power compared to a brute-force generation method. For example, since frequent item-set 1006 does not have a valid fourth item greater than 8, possible candidates are not generated and therefore the set is terminated at step 1030. The overall flow is to find the frequent item-sets from the presented data set (e.g., transactions) using said Apriori algorithm. Item-sets that correspond to transactions on cases with positive outcomes are selected as actions to take during the next business cycle to improve outcomes. In various embodiments, the winnowed data for each outcome/case type/interaction type combination is processed by the said modified Apriori algorithm, which will result in a list of 2-8 items that, when implemented, will have the greatest impact on improving the frequency of the outcome. For example, the output could appear as follows:
1) Desired Outcome=Readmission rate<0.1, Case Type=Total Knee Replacement, Interaction Type=Surgical Discharge
2) Medication Topic Time>=120,000 milliseconds
3) What to expect after surgery viewing compliance>=0.8
4) Questions Topic Visited>=2
5) External Subject Speaking>=270,000 milliseconds
6) External Subject Interest Emotion>=0.7
7) Interaction Time>=840,000 milliseconds
8) Interaction Time<=1,260,000 milliseconds
   From this data set, for the next 90 days the most impactful changes the business could take during the surgical discharge to meet the desired outcome of readmission rates less than 10% for total knee replacements are:
1) Spend at least 2 minutes talking about medications.
2) Ask the patient if they have any questions 2 or more times during the discharge.
3) Make sure that the patient is talking at least 4½ minutes, or roughly ⅓ of the total interaction time. If the patient is too quiet, use techniques to elicit verbal engagement.
4) Ensure that the patient is interested (not apathetic) in the discussion, and if not, employ techniques to gain their interest.
5) Reinforce that when they go home the patient should watch the “What to expect after surgery” educational video. Additionally, monitor compliance with this instruction after the discharge.
6) Ensure that the discharge interaction takes between 14 and 20 minutes.

Certain aspects of the present disclosure provide for an Analysis Data Store 116 of FIG. 1 capable of providing a persistent storage mechanism for data produced by the Analysis Engine 702 of FIG. 7. In various embodiments, said data analysis store records both input and output sets from each stage in an analysis pipeline. The mechanism for storage can be any appropriate mechanism for the data in question, including but not limited to, relational tables, flat files, binary trees, etc. When appropriate, the analysis data store may use external systems for storage of all or part of the data by using the External Connection Engine 110 of FIG. 1 In various embodiments, both final results and interim stage results are stored in the Analysis Data Store 116 of FIG. 1 for visualization or reporting in the Visualization Engine 118 of FIG. 1.

Certain aspects of the present disclosure provide for a Visualization Engine 118 of FIG. 1 capable of combining one or more of the outputs of the Analysis Data Store 116 of FIG. 1 and the Transactional Data Store 114 of FIG. 1 to produce reports or other visualizations from the output of the analysis engine. The visualization engine provides support for these key functions: 1) support time-series views (e.g., Trendlines, year to year comparisons, etc.) by consolidating the output of daily runs of the analysis engine; 2) support roll-up and drill-down capabilities by using taxonomy links to the attributes used in the analysis; and 3) support export to various formats, including visual formats (graphs, heat maps, etc.) as well as tabular (row and columns of data) formats. In various embodiments, the specific implementation of the visualization engine may be a custom-written application, link to an external data visualization tool through the external connection engine, combinations thereof, or the like.

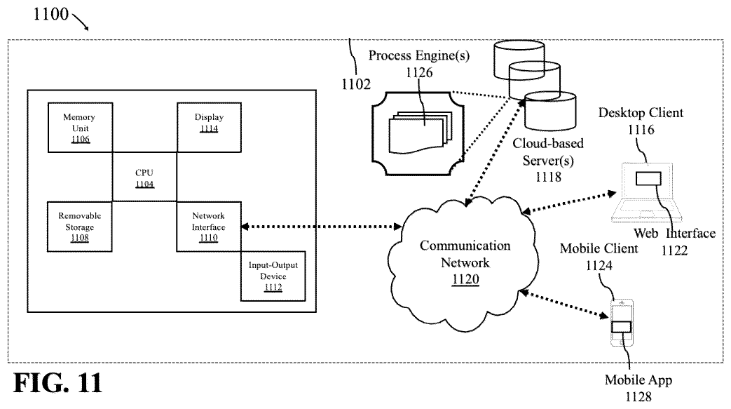

Referring now to FIG. 11, a block diagram 1100 of a general-purpose computer implemented system 1102 configured for process mining of healthcare data to enable process discovery, conformance, performance, and organization analyses in the provision of high-quality patient care management, patient education, patient engagement, and care coordination is shown. In accordance with various aspects of the present disclosure, computer system 1102 may comprise systems and/or sub-systems, including at least one microprocessor 1104, memory unit (e.g., ROM) 1106, removable storage device (e.g., RAM) 1108, fix/removable storage device(s), network interface 1110, input-output (I/O) device 1112, display 1114, and keyboard 1116. In various embodiments, the general-purpose computing system 1102 may serve as a client-server system enabling user access to the automated and integrated system and methods, locally or as a desktop client 1116, of a distributed computing platform or back-end or cloud-based server 1118 via a communication network 1120. In various embodiments, the general-purpose computing system 1102 may serve as a client-serve enabling user access to the automated and integrated system and methods, locally or as a client, of a distributed computing platform or back-end server, accessible via a computing device having an administrative/navigator web interface 1122. In various embodiments, web interface 1122 may comprise one or more dashboards. In various embodiments, the computer system in accordance with the present disclosure may comprise systems and/or sub-systems, including one or more desktop client 1116, a laptop, tablet, portable, or mobile phone 1124 computing device. In various embodiments, the general-purpose computer implemented system 1102 may comprise at least one process engine 1126, including but not limited to one or more disclosed, definition engine, process execution engine, ingestion engine, external connection engine, analysis engine, transaction data store, analysis data store, and a visualization engine. In various embodiments, the general-purpose computing system 1102 may serve as a client-server system enabling user access to the automated and integrated system and methods, locally or as a client, of a distributed computing platform or back-end server, accessible via a mobile client 1124 through a mobile application 1128. The multimodal process mining system enables the capture and analyses of multimodal data relating to complex clinical workflows, process model extraction of patient care events, monitoring deviations by comparing model and data collection, social network or organizational mining, automated simulation of models, model extension, case prediction, and recommendations to improve one or more operational consistency, efficiency, precision, accuracy, analytics, costs, business, or process outcomes.

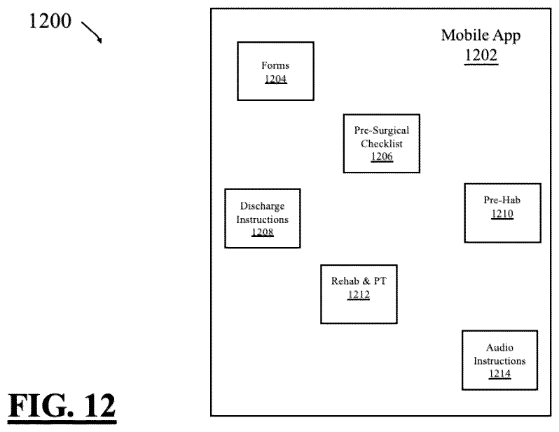

Referring now to FIG. 12, a block diagram 1200 of a mobile application is shown. In accordance with various aspects of the present disclosure, a mobile application (“app”) 1202 may be embodied as mobile app 1128 of FIG. 11. Mobile app 1202 may enable a patient, care giver or healthcare provider access to an automated or integrated system as described above. A provider can use mobile app 1202 to select, configure, or use at least one function, including but not limited to, a diagnostic, intervention, prescription, education content, recommendation, calendar, scheduling, capture one or more multimodal data, forms 1204, pre-surgical checklist 1206, discharge instructions 1208, pre-habilitation 1210 and physical therapy instructions 1212, audio instructions 1214, relating to high-quality patient care management, patient education, patient engagement, and care coordination. The app may enable a provider, doctor, nurse, healthcare manager, or patient to communicate, send, receive, or view the results of a process discovery, conformance, performance, organization analyses, improved operational consistency, efficiency, precision, accuracy, analytics, costs, business, or process outcomes.

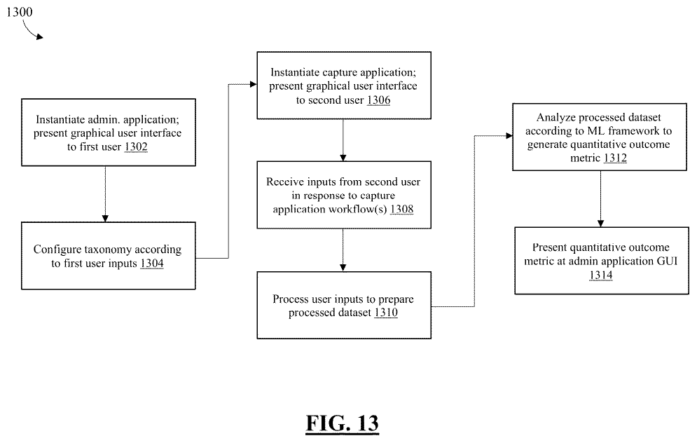

Referring now to FIG. 13, a process-flow diagram of a computer-implement method 1300 is shown. In accordance with certain aspects of the present disclosure, method 1300 may be embodied within one or more system routines or operations of automated and integrated system 100 for process mining of healthcare data to enable process discovery, conformance, performance, and organization analyses in the provision of high-quality patient care management, patient education, patient engagement, and care coordination, as shown and described in FIG. 1. In accordance with certain aspects of the present disclosure, method 1300 may be embodied in one or more system, apparatus and/or computer-program product embodied in one or more processor-executable instructions stored on at least one non-transitory computer readable storage medium. In accordance with certain aspects of the present disclosure, method 1300 may comprise one or more steps or operations for presenting (e.g., with a first processor communicably engaged with a display of a first client device) a first graphical user interface to a first end user (Step 1302). In certain embodiments, the first graphical user interface is associated with an administrator application configured to enable the first end user to configure at least one taxonomy comprising a plurality of data types for at least one user workflow. In certain embodiments, one or more aspects of the at least one workflow may be embodied as a capture application, as described above. Method 1300 may proceed by executing one or more steps or operations for configuring (e.g., with the first processor) the at least one taxonomy in response to one or more user-generated inputs from the first end user at the first graphical user interface (Step 1304). In certain embodiments, the taxonomy may comprise a hierarchical structure for organization and classification purposes related to the at least one workflow. Method 1300 may proceed by executing one or more steps or operations for instantiating the capture application (e.g., as described herein) and presenting (e.g., with a second processor communicably engaged with a display of a second client device) a second graphical user interface to a second end user (Step 1306). In certain embodiments, the second graphical user interface may comprise one or more interface elements associated with at least one user workflow for the capture application, as described above. Method 1300 may proceed by executing one or more steps or operations for receiving (e.g., with the second processor via the second client device) a plurality of user-generated inputs from the second end user in response to the at least one user workflow (Step 1308). In certain embodiments, the plurality of user-generated inputs may comprise at least one input via the second client device and at least one voice input via a microphone of the second client device. Method 1300 may proceed by executing one or more steps or operations for processing (e.g., with one or both of the first processor and the second processor) the plurality of user-generated inputs according to at least one data processing framework to prepare a processed dataset (Step 1310). In certain embodiments, the processed dataset may comprise at least one audio file. The at least one audio file may comprise the at least one voice input. In certain embodiments, the at least one data processing framework comprises a speech-to-text engine configured to convert the at least one audio file to text data.

In accordance with certain aspects of the present disclosure, method 1300 may proceed by executing one or more steps or operations for analyzing (e.g., with one or both of the first processor and the second processor) the processed dataset according to at least one machine learning framework (Step 1312). In accordance with certain embodiments, the at least one machine learning framework may comprise a clustering algorithm configured to identify one or more attributes from the processed dataset and cluster two or more datapoints from the processed dataset according to the one or more attributes. In certain embodiments, the clustering algorithm comprises a k-means++ clustering algorithm. In accordance with certain embodiments, the at least one machine learning framework may comprise a classification algorithm configured to analyze an output of the clustering algorithm to classify the one or more attributes according to a predictive strength for at least one quantitative outcome for the at least one user workflow. In certain embodiments, the classification algorithm comprises a naïve Bayesian algorithm. In accordance with certain embodiments, the at least one machine learning framework comprises at least one Apriori algorithm configured to analyze an output of the classification algorithm to generate at least one quantitative outcome metric for the at least one user workflow. Method 1300 may proceed by executing one or more steps or operations for presenting (e.g., with the first processor) the at least one quantitative outcome metric at the display of the first client device to the first end user (Step 1314).

In accordance with certain aspects of the present disclosure, method 1300 may optionally comprise one or more steps or operations for generating (e.g., with the first processor) one or more recommendations for modifying or configuring one or more steps of the at least one user workflow according to the at least one quantitative outcome metric. Method 1300 may further comprise one or more steps or operations for algorithmically modifying or configuring (e.g., with the first processor) the one or more steps of the at least one user workflow according to the one or more recommendations. In accordance with certain aspects of the present disclosure, method 1300 may optionally comprise one or more steps or operations for analyzing (e.g., according to the at least one data processing framework) the at least one audio file to determine one or more speaker identity from the at least one voice input. In certain embodiments, the at least one data processing framework comprises a speaker identification engine. In accordance with certain aspects of the present disclosure, method 1300 may optionally comprise one or more steps or operations for analyzing (e.g., according to the at least one data processing framework) the at least one audio file to determine one or more degrees of sentiment for the one or more speaker identity. Method 1300 may optionally comprise one or more steps or operations for presenting (e.g., via the display of the first client device) the one or more recommendations for modifying or configuring the one or more steps of the at least one user workflow according to the at least one quantitative outcome metric. Method 1300 may optionally comprise one or more steps or operations for rendering (e.g., with the first processor via the display of the first client device) at least one graphical data visualization comprising one or more outputs of the at least one data processing framework and the at least one machine learning framework.

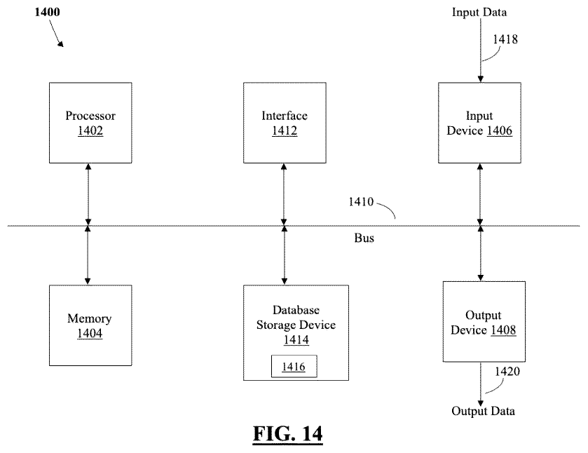

Referring now to FIG. 14, a processor-implemented computing device in which one or more aspects of the present disclosure may be implemented is shown. According to an embodiment, a processing system 1400 may generally comprise at least one processor 1402, or processing unit or plurality of processors, memory 1404, at least one input device 1406 and at least one output device 1408, coupled together via a bus or group of buses 1410. In certain embodiments, input device 1406 and output device 1408 could be the same device. An interface 1412 can also be provided for coupling the processing system 1400 to one or more peripheral devices, for example interface 1412 could be a PCI card or PC card. At least one storage device 1414 which houses at least one database 1416 can also be provided. The memory 1404 can be any form of memory device, for example, volatile or non-volatile memory, solid state storage devices, magnetic devices, etc. The processor 1402 could comprise more than one distinct processing device, for example to handle different functions within the processing system 1400. Input device 1406 receives input data 1418 and can comprise, for example, a keyboard, a pointer device such as a pen-like device or a mouse, audio receiving device for voice-controlled activation such as a microphone, data receiver or antenna such as a modem or wireless data adaptor, data acquisition card, etc. Input data 1418 could come from different sources, for example keyboard instructions in conjunction with data received via a network. Output device 1408 produces or generates output data 1420 and can comprise, for example, a display device or monitor in which case output data 1420 is visual, a printer in which case output data 1420 is printed, a port for example a USB port, a peripheral component adaptor, a data transmitter or antenna such as a modem or wireless network adaptor, etc. Output data 1420 could be distinct and derived from different output devices, for example a visual display on a monitor in conjunction with data transmitted to a network. A user could view data output, or an interpretation of the data output, on, for example, a monitor or using a printer. The storage device 1414 can be any form of data or information storage means, for example, volatile or non-volatile memory, solid state storage devices, magnetic devices, etc.

In use, the processing system 1400 is adapted to allow data or information to be stored in and/or retrieved from, via wired or wireless communication means, at least one database 1416. The interface 1412 may allow wired and/or wireless communication between the processing unit 1402 and peripheral components that may serve a specialized purpose. In general, the processor 1402 can receive instructions as input data 1418 via input device 1406 and can display processed results or other output to a user by utilizing output device 1408. More than one input device 1406 and/or output device 1408 can be provided. It should be appreciated that the processing system 1400 may be any form of terminal, server, specialized hardware, or the like.

It is to be appreciated that the processing system 1400 may be a part of a networked communications system. Processing system 1400 could connect to a network, for example the Internet or a WAN. Input data 1418 and output data 1420 could be communicated to other devices via the network. The transfer of information and/or data over the network can be achieved using wired communications means or wireless communications means. A server can facilitate the transfer of data between the network and one or more databases. A server and one or more databases provide an example of an information source.

Thus, the processing computing system environment 1400 illustrated in FIG. 14 may operate in a networked environment using logical connections to one or more remote computers. The remote computer may be a personal computer, a server, a router, a network PC, a peer device, or other common network node, and typically includes many or all of the elements described above.

It is to be further appreciated that the logical connections depicted in FIG. 14 include a local area network (LAN) and a wide area network (WAN) but may also include other networks such as a personal area network (PAN). Such networking environments are commonplace in offices, enterprise-wide computer networks, intranets, and the Internet. For instance, when used in a LAN networking environment, the computing system environment 1400 is connected to the LAN through a network interface or adapter. When used in a WAN networking environment, the computing system environment typically includes a modem or other means for establishing communications over the WAN, such as the Internet. The modem, which may be internal or external, may be connected to a system bus via a user input interface, or via another appropriate mechanism. In a networked environment, program modules depicted relative to the computing system environment 1400, or portions thereof, may be stored in a remote memory storage device. It is to be appreciated that the illustrated network connections of FIG. 14 are exemplary and other means of establishing a communications link between multiple computers may be used.

FIG. 14 is intended to provide a brief, general description of an illustrative and/or suitable exemplary environment in which embodiments of the below described present invention may be implemented. FIG. 14 is an example of a suitable environment and is not intended to suggest any limitation as to the structure, scope of use, or functionality of an embodiment of the present invention. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

As will be appreciated by one of skill in the art, the present invention may be embodied as a method (including, for example, a computer-implemented process, a business process, and/or any other process), apparatus (including, for example, a system, machine, device, computer program product, and/or the like), or a combination of the foregoing. Accordingly, embodiments of the present invention may take the form of an entirely hardware embodiment, an entirely software embodiment (including firmware, resident software, micro-code, etc.), or an embodiment combining software and hardware aspects that may generally be referred to herein as a “system.” Furthermore, embodiments of the present invention may take the form of a computer program product on a computer-readable medium having computer-executable program code embodied in the medium.

Any suitable transitory or non-transitory computer readable medium may be utilized. The computer readable medium may be, for example but not limited to, an electronic, magnetic, optical, electromagnetic, infrared, or semiconductor system, apparatus, or device. More specific examples of the computer readable medium include, but are not limited to, the following: an electrical connection having one or more wires; a tangible storage medium such as a portable computer diskette, a hard disk, a random-access memory (RAM), a read-only memory (ROM), an erasable programmable read-only memory (EPROM or Flash memory), a compact disc read-only memory (CD-ROM), or other optical or magnetic storage device.

In the context of this document, a computer readable medium may be any medium that can contain, store, communicate, or transport the program for use by or in connection with the instruction execution system, apparatus, or device. The computer usable program code may be transmitted using any appropriate medium, including but not limited to the Internet, wireline, optical fiber cable, radio frequency (RF) signals, or other mediums.

Computer-executable program code for carrying out operations of embodiments of the present invention may be written in an aspect oriented, scripted, or unscripted programming language such as Java, Perl, Smalltalk, C++, or the like. However, the computer program code for carrying out operations of embodiments of the present invention may also be written in conventional procedural programming languages, such as the “C” programming language or similar programming languages.

Embodiments of the present invention are described above with reference to flowchart illustrations and/or block diagrams of methods, apparatus (systems), and computer program products. It will be understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general-purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks.

These computer-executable program code portions may also be stored in a computer-readable memory that can direct a computer or other programmable data processing apparatus to function in a particular manner, such that the code portions stored in the computer readable memory produce an article of manufacture including instruction mechanisms which implement the function/act specified in the flowchart and/or block diagram block(s).

The computer-executable program code may also be loaded onto a computer or other programmable data processing apparatus to cause a series of operational phases to be performed on the computer or other programmable apparatus to produce a computer-implemented process such that the code portions which execute on the computer or other programmable apparatus provide phases for implementing the functions/acts specified in the flowchart and/or block diagram block(s). Alternatively, computer program implemented phases or acts may be combined with operator or human implemented phases or acts to carry out an embodiment of the invention.

As the phrase is used herein, a processor may be “configured to” perform a certain function in a variety of ways, including, for example, by having one or more general-purpose circuits perform the function by executing computer-executable program code embodied in computer-readable medium, and/or by having one or more application-specific circuits perform the function.

Embodiments of the present invention are described above with reference to flowcharts and/or block diagrams. It will be understood that phases of the processes described herein may be performed in orders different than those illustrated in the flowcharts. In other words, the processes represented by the blocks of a flowchart may, in some embodiments, be in performed in an order other than the order illustrated, may be combined, or divided, or may be performed simultaneously. It will also be understood that the blocks of the block diagrams illustrated, in some embodiments, merely conceptual delineations between systems and one or more of the systems illustrated by a block in the block diagrams may be combined or share hardware and/or software with another one or more of the systems illustrated by a block in the block diagrams. Likewise, a device, system, apparatus, and/or the like may be made up of one or more devices, systems, apparatuses, and/or the like. For example, where a processor is illustrated or described herein, the processor may be made up of a plurality of microprocessors or other processing devices which may or may not be coupled to one another. Likewise, where a memory is illustrated or described herein, the memory may be made up of a plurality of memory devices which may or may not be coupled to one another.

While certain exemplary embodiments have been described and shown in the accompanying drawings, it is to be understood that such embodiments are merely illustrative of, and not restrictive on, the broad invention, and that this invention is not limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

References and citations to other documents, such as patents, patent applications, patent publications, journals, books, papers, web contents, have been made throughout this disclosure. All such documents are hereby incorporated herein by reference in their entirety for all purposes.

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof

What is claimed is:

1. A computer-implemented method comprising:
presenting, with a first processor communicably engaged with a display of a first client device, a first graphical user interface to a first end user, wherein the first graphical user interface comprises one or more interface elements configured to enable the first end user to configure at least one taxonomy comprising a plurality of data types for at least one user workflow;
configuring, with the first processor, the at least one taxonomy in response to one or more user-generated inputs from the first end user at the first graphical user interface;
presenting, with a second processor communicably engaged with a display of a second client device, a second graphical user interface to a second end user, wherein the second graphical user interface comprises one or more interface elements associated with the at least one user workflow;
receiving, with the second processor via the second client device, a plurality of user-generated inputs from the second end user in response to the at least one user workflow, wherein the plurality of user-generated inputs comprises at least one input via the second client device and at least one voice input via a microphone of the second client device;
processing, with one or both of the first processor and the second processor, the plurality of user-generated inputs according to at least one data processing framework to prepare a processed dataset comprising at least one audio file comprising the at least one voice input, wherein the at least one data processing framework comprises a speech-to-text engine configured to convert the at least one audio file to text data;
analyzing, with one or both of the first processor and the second processor, the processed dataset according to at least one machine learning framework,
wherein the at least one machine learning framework comprises a clustering algorithm configured to identify one or more attributes from the processed dataset and cluster two or more datapoints from the processed dataset according to the one or more attributes,
wherein the at least one machine learning framework comprises a classification algorithm configured to analyze an output of the clustering algorithm to classify the one or more attributes according to a predictive strength for at least one quantitative outcome for the at least one user workflow,
wherein the at least one machine learning framework comprises at least one Apriori algorithm configured to analyze an output of the classification algorithm to generate at least one quantitative outcome metric for the at least one user workflow; and
presenting, with the first processor, the at least one quantitative outcome metric at the display of the first client device to the first end user.

2. The computer-implemented method of claim 1 further comprising generating, with the first processor, one or more recommendations for modifying or configuring one or more steps of the at least one user workflow according to the at least one quantitative outcome metric.

3. The computer-implemented method of claim 2 further comprising algorithmically modifying or configuring, with the first processor, the one or more steps of the at least one user workflow according to the one or more recommendations.

4. The computer-implemented method of claim 1 wherein the classification algorithm comprises a naïve Bayesian algorithm.

5. The computer-implemented method of claim 1 wherein the clustering algorithm comprises a k-means++ clustering algorithm.

6. The computer-implemented method of claim 1 further comprising analyzing, according to the at least one data processing framework, the at least one audio file to determine one or more speaker identity from the at least one voice input, wherein the at least one data processing framework comprises a speaker identification engine.

7. The computer-implemented method of claim 6 further comprising analyzing, according to the at least one data processing framework, the at least one audio file to determine one or more degrees of sentiment for the one or more speaker identity.

8. The computer-implemented method of claim 2 further comprising presenting, via the display of the first client device, the one or more recommendations for modifying or configuring the one or more steps of the at least one user workflow according to the at least one quantitative outcome metric.

9. The computer-implemented method of claim 1 further comprising rendering, with the first processor via the display of the first client device, at least one graphical data visualization comprising one or more outputs of the at least one data processing framework and the at least one machine learning framework.

10. A computer-implemented system comprising:
a client device comprising an input device, a microphone and a display; and
a server communicably engaged with the client device, the server comprising a processor and a non-transitory computer-readable medium communicably engaged with the processor, wherein the non-transitory computer-readable medium comprises one or more processor-executable instructions stored thereon that, when executed, command the processor to perform one or more operations, the one or more operations comprising:
configuring at least one taxonomy comprising a plurality of data types for at least one user workflow;
rendering an instance of a data capture application at the client device;
presenting a graphical user interface of the data capture application to an end user at the display of the client device, wherein the graphical user interface comprises one or more interface elements associated with the at least one user workflow;
receiving a plurality of user-generated inputs from the end user according to the at least one user workflow, wherein the plurality of user-generated inputs comprises at least one input via the input device and at least one voice input via the microphone;
processing the plurality of user-generated inputs according to at least one data processing framework to prepare a processed dataset comprising at least one audio file comprising the at least one voice input, wherein the at least one data processing framework comprises a speech-to-text engine configured to convert the at least one audio file to text data;
analyzing the processed dataset according to at least one machine learning framework,
wherein the at least one machine learning framework comprises a clustering algorithm configured to identify one or more attributes from the processed dataset and cluster two or more datapoints from the processed dataset according to the one or more attributes,
wherein the at least one machine learning framework comprises a classification algorithm configured to analyze an output of the clustering algorithm to classify the one or more attributes according to a predictive strength for at least one quantitative outcome for the at least one user workflow,
wherein the at least one machine learning framework comprises at least one Apriori algorithm configured to analyze an output of the classification algorithm to generate at least one quantitative outcome metric for the at least one user workflow; and
presenting the at least one quantitative outcome metric at the display of the client device to the end user.

11. The computer-implemented system of claim 10 wherein the one or more operations further comprise generating one or more recommendations for modifying or configuring one or more steps of the at least one user workflow according to the at least one quantitative outcome metric.

12. The computer-implemented system of claim 11 wherein the one or more operations further comprise algorithmically modifying or configuring the one or more steps of the at least one user workflow according to the one or more recommendations.

13. The computer-implemented system of claim 10 wherein the classification algorithm comprises a naïve Bayesian algorithm.

14. The computer-implemented system of claim 10 wherein the clustering algorithm comprises a k-means++ clustering algorithm.

15. The computer-implemented system of claim 10 wherein the one or more operations further comprise analyzing, according to the at least one data processing framework, the at least one audio file to determine one or more speaker identity from the at least one voice input, wherein the at least one data processing framework comprises a speaker identification engine.

16. The computer-implemented system of claim 15 wherein the one or more operations further comprise analyzing, according to the at least one data processing framework, the at least one audio file to determine one or more degrees of sentiment for the one or more speaker identity.

17. The computer-implemented system of claim 11 wherein the one or more operations further comprise presenting, via the display of the client device, the one or more recommendations for modifying or configuring the one or more steps of the at least one user workflow according to the at least one quantitative outcome metric.

18. The computer-implemented system of claim 10 wherein the one or more operations further comprise rendering, at the display of the client device, at least one graphical data visualization comprising one or more outputs of the at least one data processing framework and the at least one machine learning framework.

19. The computer-implemented system of claim 10 further comprising a transactional data store communicably engaged with the server, wherein the transactional data store is configured to receive and store the plurality of user-generated inputs, the processed dataset, and one or more outputs from the at least one machine learning framework.

20. A non-transitory computer-readable medium with one or more processor-executable instructions stored thereon that, when executed, command one or more processors to perform one or more operations, the one or more operations comprising:
configuring at least one taxonomy comprising a plurality of data types for at least one user workflow;
rendering an instance of a data capture application at a client device;
presenting a graphical user interface of the data capture application to an end user at a display of the client device, wherein the graphical user interface comprises one or more interface elements associated with the at least one user workflow;
receiving a plurality of user-generated inputs from the end user according to the at least one user workflow, wherein the plurality of user-generated inputs comprises at least one input via an input device of the client device and at least one voice input via a microphone of the client device;
processing the plurality of user-generated inputs according to at least one data processing framework to prepare a processed dataset comprising at least one audio file comprising the at least one voice input, wherein the at least one data processing framework comprises a speech-to-text engine configured to convert the at least one audio file to text data;
analyzing the processed dataset according to at least one machine learning framework,
wherein the at least one machine learning framework comprises a clustering algorithm configured to identify one or more attributes from the processed dataset and cluster two or more datapoints from the processed dataset according to the one or more attributes,
wherein the at least one machine learning framework comprises a classification algorithm configured to analyze an output of the clustering algorithm to classify the one or more attributes according to a predictive strength for at least one quantitative outcome for the at least one user workflow,
wherein the at least one machine learning framework comprises at least one Apriori algorithm configured to analyze an output of the classification algorithm to generate at least one quantitative outcome metric for the at least one user workflow; and
presenting the at least one quantitative outcome metric at the display of the client device to the end user.
